(19) United States
(12) Patent Application Publication
(10) Pub. No.: US 2008/0131522 A1
(43) Pub. Date: Jun. 5, 2008

(54) Use of placental biomaterial for ocular surgery

(76) Inventors: Qing Liu, Hillsborough, NJ (US); Cynthia Ray, Flemington, NJ (US)

Correspondence Address: JONES DAY, 222 EAST 41ST ST, NEW YORK, NY, 10017, US

(21) Appl. No.: 11/906,961

(22) Filed: Oct. 3, 2007

Related U.S. Application Data
(60) Provisional application No. 60/849,319, filed on Oct. 3, 2006.

(51) Int. Cl. A61K 35/48

(52) U.S. Cl. 424/583

(57) ABSTRACT

The present application is directed to a method of performing ocular surgery using biomaterial from placenta and/or umbilical cord, wherein the biomaterial is contacted with a tissue of the eye, or tissue surrounding the eye, affected by the surgery. The biomaterial is also used in surgery performed to repair trauma to the eye, whether deliberately or accidentally caused. The eye surgery can be, e.g., glaucoma surgery, oculoplastic surgery, cataract surgery, vitreo-retinal surgery, refractive surgery, retinal surgery, eye muscle surgery, or the like.

### 1. FIELD OF THE INVENTION

The present invention relates to the use of umbilical cord biomaterial, such as umbilical cord membrane, either in unprocessed or processed form, or placental biomaterial, such as amniotic membrane, either in unprocessed or processed form, as an adjunct to ocular surgeries.

### 2. BACKGROUND OF THE INVENTION

Certain compositions, such as cadaveric tissues, have been used in the repair or treatment of discontinuities in the eye resulting from, or caused as an adjunct to, ocular surgery. See, e.g., Brandt, Arch. Opthalmol. 111:1436-1439 (1993). A need exists, however, for additional such compositions that exhibit increased biocompatibility and decreased immunogenicity.

### 3. SUMMARY OF THE INVENTION

The present invention provides methods of performing ocular surgery comprising the use of placental biomaterial, such as amniotic membrane, and/or umbilical cord biomaterial, particularly umbilical cord membrane, as, e.g., a patch, graft, or wound healing material. In preferred embodiments, the umbilical cord biomaterial comprises Wharton's jelly.

In one aspect, the invention provides a method of performing an ocular surgical procedure that involves a structure of an eye or tissue adjacent to an eye, and contacting said structure with placental biomaterial and/or umbilical cord biomaterial. In a specific embodiment, the umbilical cord biomaterial is umbilical cord membrane. In a more specific, preferred, embodiment, the umbilical cord membrane comprises Wharton's jelly. In another more specific embodiment, the umbilical cord membrane, optionally including Wharton's jelly, is dried. In another specific embodiment, said contacting comprises using said umbilical cord membrane as a patch over a discontinuity in said structure. In another specific embodiment, said discontinuity is a fistula. In a more specific embodiment, said discontinuity is an incision made during said surgical procedure. In another specific embodiment, said ocular surgical procedure is creation of a scleral buckle. In another specific example, said ocular surgical procedure is a glaucoma surgery. In a more specific example, said ocular surgical procedure is implantation of a drainage implant or tube shunt. In a more specific example, said umbilical cord membrane additionally contacts said drainage implant or said tube shunt. In another specific embodiment, said drainage implant is a silicone tube. In a preferred embodiment, said umbilical cord biomaterial substantially prevents deterioration of said silicone tube for at least six months.

In various specific embodiments, said glaucoma surgery is trebeculoplasty, iridotomy, iridactomy, a filtering surgery, anterior sclerotomy, sclerostomy, penetrating trabeculotomy, non-penetrating trabeculotomy, trepanotrabeculectomy, goniotomy, cyclotomy, cycloanemization, cyclocryotherapy, cyclocryopexy, iridenclesis, iridocyclectomy, iridosclerotomy, corenclisis or goniocurretage.

In another embodiment of the method, the ocular surgical procedure is cataract surgery. In a specific embodiment, said cataract surgery is phacoemulsification of a cataract in said eye. In a more specific embodiment, said contacting comprises repair of a phacoemulsification burn. In another specific embodiment, said ocular surgical surgery is a trabulectomy.

In another embodiment of the method, said ocular surgical procedure is implantation of an orbital implant.

In another embodiment of the method, said ocular surgical procedure changes the shape of the cornea or is refractive surgery. In a specific embodiment, said ocular surgical procedure is photorefractive keratectomy (PRK), laser-assisted sub-epithelial keratectomy (LASEK) or laser-assisted in situ keratomileusis (LASIK). In another specific embodiment, said ocular surgical procedure is automated lamellar keratoplasty (ALK), laser thermal keratoplasty (LTK), or conductive keratoplasty (CK).

In another embodiment of the method, said ocular surgical procedure is a vitreoretinal surgery. In a specific embodiment, said vitreoretinal surgery is an anterior vitrectomy, pars plana vitrectomy, ignipuncture, macular hole repair, partial lamellar sclerouvectomy, or posterior sclerotomy.

In another embodiment of the method, said ocular surgical procedure is retinal surgery.

In another embodiment of the method, said ocular surgical procedure is an eye muscle surgery.

In another embodiment of the method, said ocular surgical procedure is an oculoplastic surgery. In specific embodiments, said oculoplastic surgery is browplasty, eyelid reconstruction, blepharoplasty, ectropion repair, entropion repair, canthal resection, canthectomy, cantholysis, canthopexy, canthoplasty, canthorrhapy, canthotomy, canaliculodacryocystostomy, canaliculotomy, dacryoadenectomy, dacryocystectomy, dacryocystorhiostomy, dacryocystostomy, enucleation of the eye, evisceration or exenteration. In a specific embodiment, said ocularplastic surgery is repair of an exposed orbital implant.

In another specific embodiment, said ocular surgical procedure is corneal surgery.

In various other specific embodiments, said ocular surgical procedure is ciliarotomy, ciliectomy, ciliotomy, corectomedialysis, corectomy, corelysis, coremorphosis, coreplasty, coreoplasty, pupillomydriasis, cyclectomy, cyclectomy, iridectomesodialysis, iridodialysis, iridesis or iridocorneosclerectomy.

In another specific embodiment, the surgery is surgery to correct, repair or ameliorate trauma to the eye, e.g., trauma caused by, or associated with, a discontinuity in the eye. The trauma can be caused outside a surgical context, e.g., can be accidentally caused.

In another specific embodiment of the above methods, said placental biomaterial or umbilical cord biomaterial is substantially dry (<20% water by weight) prior to said contacting. In another specific embodiment, said placental biomaterial or umbilical cord biomaterial is between about 100 microns and about 1000 microns in thickness. In another specific embodiment, said placental biomaterial or umbilical cord biomaterial is about 400 microns in thickness. In another specific embodiment, said placental biomaterial or umbilical cord biomaterial is about 50 to about 100 microns in thickness. In another specific embodiment, said placental biomaterial or umbilical cord biomaterial is decellularized. In another specific embodiment, the umbilical cord biomaterial comprises Wharton's jelly. In another specific embodiment, said placental biomaterial or umbilical cord biomaterial is hydrated prior to said contacting. In another specific embodiment, said placental biomaterial or umbilical cord biomaterial is from about 0.25 cm×2.0 cm to about 0.5 cm×4.0 cm in size.

As used herein, “placental biomaterial” means a material made from placental cells, either in vivo or in vitro, that can act as a substrate or scaffold for epithelialization, and has reduced immunogenicity relative to non-placental tissue, and includes, but is not limited to, the placenta, including individually or collectively the amniotic membrane, chorion, or an extracellular matrix (ECM) made from, or contributed to by, placental cells. The term includes placental material that has been decellularized, or ECM, made from, or contributed to by, placental cells, that is decellularized.

As used herein, “umbilical cord biomaterial” means a material made from umbilical cord cells, either in vivo or in vitro, that can act as a substrate or scaffold for epithelialization, and has reduced immunogenicity relative to non-placental tissue, and includes, but not limited to, e.g., whole umbilical cord, dried whole umbilical cord, umbilical cord membrane (with or without vessels, and with or without Wharton's jelly), dried umbilical cord membrane, umbilical cord membrane laminated with a second material, etc. The term includes umbilical cord material that has been decellularized or ECM, made from, or contributed to by, umbilical cord cells, that is decellularized.

### 4. BRIEF DESCRIPTION OF THE FIGURES

FIGS. 1A and 1B depict the effect of radiation dose on water uptake [⋄, (Ww−Wd)/Wd*100] and equilibrium water content [symbol] (Ww−Wd)/Ww*100] for human umbilical cord biomaterial incubated for 10 (A) and 20 (B) days in 1% D-cell solution. Ww=weight when wet; Wd=weight when dry.

FIGS. 2A and 2B depict a comparison of the change in thickness [symbol] and water uptake (▪) during rehydration of gamma sterilized human umbilical cord biomaterial incubated for 10 (A) and 20 (B) days in 1% D-cell solution. Error bars indicate standard deviation.

FIG. 3 depicts a comparison of the denaturation temperature of rehydrated human umbilical cord membrane that had previously been incubated in 1% D-cell solution for 10 or 20 days. There is essentially no difference between the different incubation times and there is a linear decrease in denaturation temperature with increasing radiation dose.

FIG. 4A depicts a suture pull-out assay apparatus with an lower grip 10 holding vellum paper 11 to which a 1×2 cm section of umbilical cord biomaterial 12 is glued, and an upper grip 13 attached to a suture 14 that passes through the umbilical cord biomaterial. Force was applied upwards through the suture at approximately 12.7 mm/min. FIG. 4B depicts results of a comparison of the human umbilical cord biomaterial (HUC) and dried human amniotic membrane pull-out resistance in Newtons (N).

### 5. DETAILED DESCRIPTION OF THE INVENTION

The present invention provides methods of performing ocular surgery, and repairing discontinuities in the eye, or surrounding tissue, that are caused by, or adjunct to, ocular surgery, comprising using placental biomaterial or umbilical cord biomaterial. Such placental biomaterial or umbilical cord biomaterial can be any biomaterial that can act as a substrate or scaffold for epithelialization, including, without limitation, amniotic membrane, chorion, umbilical cord membrane, Wharton's jelly, and the like. In preferred embodiments, said using comprises, e.g., contacting a discontinuity with, placental biomaterial or umbilical cord biomaterial.

### 5.1. Umbilical Cord Membrane and Umbilical Cord Biomaterial

### 5.1.1. Description

The present invention provides for the use of umbilical cord biomaterial in ocular surgery. The umbilical cord membrane may be from any mammalian umbilical cord, but is preferably from a human umbilical cord. The umbilical cord biomaterial, in certain embodiments, can be used as taken from the placenta, that is, comprising Wharton's jelly and cells associated with the umbilical cord membrane. In another embodiment, the umbilical cord biomaterial comprises umbilical cord membrane and Wharton's jelly, optionally also comprising cells naturally associated with the umbilical cord membrane and Wharton's jelly. In a specific embodiment in which the cells associated with the umbilical cord membrane are not removed, the cells can be killed, e.g., by freezing, prior to use in ocular surgery. Generally, the umbilical cord membrane can be used fresh from collection from the umbilical cord, or can be frozen or cryopreserved and thawed prior to use. In preferred embodiments, the umbilical cord biomaterial comprises Wharton's jelly and is substantially dry (<20% water by weight).

In certain other embodiments, the invention provides for the use of umbilical cord biomaterial in ocular surgery, wherein the biomaterial is processed (e.g., dried) umbilical cord biomaterial. The umbilical cord biomaterial is derived from a mammalian umbilical cord or part thereof that comprises an umbilical cord membrane. The umbilical cord is a substantially tubular organ, typically 10-15 cm in length, that connects the fetus to the placenta and houses the umbilical vessels. The umbilical cord comprises a membrane that wraps around two umbilical arteries and one umbilical vein, which are contained within a ground substance known as Wharton's jelly. The main components of Wharton's jelly are proteoglycans. Wharton's jelly also contains, for example, large, stellate fibroblasts and macrophages.

The umbilical cord biomaterial used in the methods of the invention typically comprises only the umbilical cord membrane, but can also comprise Wharton's jelly and/or one or more of the umbilical vessels. In one embodiment, the umbilical cord biomaterial is an umbilical cord membrane substantially isolated from the remaining umbilical cord components (e.g., Wharton's jelly and umbilical vessels). In another embodiment, the umbilical cord biomaterial comprises an umbilical cord membrane and Wharton's jelly (that is, the ground material in which the umbilical cord vessels are contained in the intact umbilical cord) that are isolated from the remaining umbilical cord components (e.g., umbilical cord vessels). In another specific embodiment, the umbilical cord membrane biomaterial comprises the umbilical cord membrane, Wharton's jelly and one or more umbilical cord vessels. In another embodiment, the umbilical cord biomaterial comprises an isolated umbilical cord (e.g., comprising Wharton's jelly and vessels, Wharton's jelly only, or vessels only) that has been flattened into a sheet or strip. The umbilical cord membrane biomaterial can be a substantially tubular structure from which the contents (Wharton's jelly and vessels) have been removed. The biomaterial can also comprise an umbilical cord membrane that has been slit or cut for part or all of the length of the umbilical cord to expose the contents of the umbilical cord.

In a specific embodiment, the biomaterial, comprising umbilical cord membrane and/or Wharton's jelly and/or vessels) can be decellularized. In another specific embodiment, the biomaterial comprises umbilical cord membrane-associated cells or Wharton's jelly-associated cells that have been killed. In another specific embodiment, the biomaterial comprises umbilical cord membrane-associated cells or Wharton's jelly-associated cells that have been maintained in a living state.

The umbilical cord biomaterial of the invention can be derived from the umbilical cord of any mammal, for example, from equine, bovine, porcine or catarrhine sources, but is most preferably derived from human umbilical cord.

The umbilical cord biomaterial can be used in ocular surgery dry or substantially dry, i.e., 20% or less water by weight. When dry, the umbilical cord biomaterial is preferably substantially flat. The dry biomaterial may, in another embodiment, substantially retain the shape of the native umbilical cord, that is, the dry membrane may be substantially tubular. The umbilical cord biomaterial can also be shaped to assume different conformations, e.g., can be curved, cut, molded, or the like, to fit to a part of the eye or surrounding tissue.

In another preferred embodiment, the umbilical cord biomaterial has not been protease-treated, heat-denatured or artificially (e.g., chemically or radiologically) crosslinked. In another embodiment, the umbilical cord biomaterial comprises artificially crosslinked proteins, e.g., chemically or radiologically crosslinked collagen. In other embodiments, the umbilical cord biomaterial contains substantially no structural proteins that are artificially crosslinked. For example, in one embodiment, the umbilical cord biomaterial is not fixed. A preferred umbilical cord biomaterial is produced by the methods disclosed herein (see 5.1.4, below, and Examples 1 and 2).

When the umbilical cord biomaterial is substantially dry, it is typically about 0.001 g/cm2 to about 0.006 g/cm2. In a specific embodiment, a single layer of the acellular, dried umbilical cord biomaterial is approximately 50 microns to 250 microns in thickness, typically approximately 90 microns to 220 microns in thickness. In other specific embodiments, a single layer of the umbilical cord biomaterial is approximately 75-200 microns, 100-200 microns, 100-220 microns, 120-220 microns, or 150-250 microns in thickness in the dry state. In another embodiment, the average thickness of the umbilical cord biomaterial is about 157 microns (e.g., ±20%).

Generally, the umbilical cord biomaterial is sided, that is, the umbilical cord biomaterial comprises an epithelial side (from the interior of the umbilical cord), and an outer, mesothelial side (from the exterior of the umbilical cord).

Generally, the umbilical cord biomaterial is non-immunogenic.

In various embodiments, the umbilical cord biomaterial comprises particular cytokines, such as, interleukin (IL)-1b, IL-2, IL-3, IL-6, IL-7, IL-12, IL-15, IFN-α, MIP-1b, and/or MCP-1.

In one embodiment, the umbilical cord biomaterial is translucent. In other embodiments, the umbilical cord biomaterial is opaque, or is colored or dyed, e.g., permanently colored or dyed, using a medically-acceptable dyeing or coloring agent. Such an agent may be adsorbed onto the biomaterial, or the biomaterial may be impregnated or coated with such an agent. In this embodiment, any known non-toxic, non-irritating coloring agent or dye may be used. In specific embodiments, only the epithelial, or mesothelial, side of the umbilical cord biomaterial is dyed. In another embodiment, both sides of the umbilical cord biomaterial are dyed.

The umbilical cord biomaterial typically comprises of collagen (types I, III and IV; typically about 75% to about 80% of the matrix of the biomaterial), fibronectin, elastin, and may further comprise glycosaminoglycans, (GAGs, e.g., hyaluronic acid) and/or proteoglycans. Typically, laminin is not present, or is present in trace amounts (i.e., less than 0.1% of the dry weight of the biomaterial). Typically, the umbilical cord biomaterial comprises collagen types I, III, IV, V, VI and VII. In certain embodiments, the umbilical cord biomaterial can comprise non-structural components, such as, for example, one or more growth factors, e.g., platelet-derived growth factors (PDGFs), vascular-endothelial growth factor (VEGF), fibroblast growth factor (FGF), transforming growth factor- [symbol] 1, and the like. In certain embodiments, the umbilical cord biomaterial comprises growth factors such as FGF, b-FGF, EGF, IGF-1, PDGF and TGF-β. The composition of the umbilical cord biomaterial may thus be ideally suited to encourage the migration of fibroblasts and macrophages, and thus, e.g., the promotion of wound healing.

In one embodiment, the invention provides for the use, in ocular surgery, of an umbilical cord biomaterial wherein at least 50% of the dry weight of the biomaterial is collagen I. In various more specific embodiments, at least 55%, 60%, 65% or 70% of the dry weight of the biomaterial is collagen I. In another specific embodiment, the invention provides an umbilical cord biomaterial wherein at most 5% of the dry weight of the biomaterial is collagen III. In various more specific embodiments, at most 4.9%, 4.8%, 4.7%, 4.6%, 4.5%, 4.4%, 4.3%, 4.2%, 4.1%, 4.0%, 3.9%, 3.8%, 3.7%, 3.6%, 3.5%, 3.4%, 3.3%, 3.2%, 3.1%, 3.0% or 2.9% of the dry weight of the biomaterial is collagen III. In another specific embodiment, the ocular surgery uses an umbilical cord biomaterial wherein at least 4% of the dry weight of the biomaterial is collagen IV. In various more specific embodiments, at least 5%, 6%, 7%, 8%, 9%, 10% or 11% of the dry weight of said biomaterial is collagen IV. In another specific embodiment, the ocular surgery uses an umbilical cord biomaterial wherein at most 4% of the dry weight of the biomaterial is elastin. In various more specific embodiments, at most 3.8%, 3.6%, 3.4%, 3.2%, 3.0%, 2.8%, 2.6%, 2.4%, 2.2%, 2.0%, or 1.8% of the dry weight of the biomaterial is elastin. In another specific embodiment, at least 4% of the dry weight of the biomaterial is glycosaminoglycan. In various more specific embodiments, at least 4.1%, 4.2%, 4.3%, 4.4%, 4.5% or 4.6% of the dry weight of said biomaterial is glycosaminoglycan.

The umbilical cord biomaterial may be used in a single-layered format, for example, as a single-layer sheet or an un-laminated membrane. Alternatively, the umbilical cord biomaterial may be used in a double-layer or multiple-layer format, e.g., the umbilical cord biomaterial may be laminated. Lamination can provide greater stiffness and durability, for example, during the healing process. The umbilical cord biomaterial may be, for example, laminated as described below (see Section 5.1.7).

The umbilical cord biomaterial may further comprise collagen from a non-umbilical cord source. For example, one or more layers of umbilical cord biomaterial may comprise, e.g., be coated or impregnated with, or layered with, purified extracted collagen. Such collagen may be obtained, for example, from commercial sources, or may be produced according to known methods, such as those disclosed in U.S. Pat. Nos. 4,420,339, 5,814,328, and 5,436,135, the disclosures of which are hereby incorporated by reference. Such collagen can also be obtained from a placental source, including a placenta obtained from the same donor as the umbilical cord biomaterial.

The umbilical cord biomaterial can comprise one or more compounds or substances that are not present in the umbilical cord material from which the biomaterial is derived. Moreover, the umbilical cord biomaterial can comprise non-naturally-occurring amounts of one or more compounds or substances that are normally present in the umbilical cord from which the biomaterial is derived. For example, the umbilical cord biomaterial can comprise, e.g., can be impregnated with, a bioactive compound, such as those listed in Section 5.1.2, below. Such bioactive compounds include, but are not limited to, small organic molecules (e.g., drugs), antibiotics (such as, for example, Clindamycin, Minocycline, Doxycycline, Gentamycin), hormones, growth factors, anti-tumor agents, anti-fungal agents, anti-viral agents, pain medications, anti-histamines, anti-inflammatory agents, anti-infectives including but not limited to silver (such as silver salts, including but not limited to silver nitrate and silver sulfadiazine), elemental silver, antibiotics, bactericidal enzymes (such as lysozyme), wound healing agents (such as cytokines including but not limited to PDGF, TGF; thymosin), hyaluronic acid as a wound healing agent, wound sealants (such as fibrin with or without thrombin), cellular attractant and scaffolding reagents (such as added fibronectin) and the like. In a specific example, the umbilical cord biomaterial may be impregnated with at least one growth factor, for example, fibroblast growth factor, epithelial growth factor, etc. The biomaterial may also be impregnated with small organic molecules such as specific inhibitors of particular biochemical processes e.g., membrane receptor inhibitors, kinase inhibitors, growth inhibitors, anticancer drugs, antibiotics, etc. Impregnating the umbilical cord biomaterial with a bioactive compound may be accomplished, e.g., by immersing the biomaterial in a solution of the bioactive compound of the desired concentration for a time sufficient to allow the biomaterial to absorb and to equilibrate with the solution. In a specific embodiment, the biomaterial so impregnated is a dried biomaterial, and the solution partially or fully re-hydrates the biomaterial, compared to an umbilical cord or umbilical cord membrane in vivo. In another embodiment, the biomaterial is impregnated prior to drying the biomaterial, e.g., to substantial dryness. In certain other embodiments, in which unprocessed umbilical cord membrane is used in ocular surgery, the umbilical cord membrane can comprise, e.g., be impregnated with, any of the compounds described above and/or in Section 5.1.2.

In other embodiments, the umbilical cord biomaterial, or umbilical cord membrane, may be combined with a hydrogel to form a composite. The use of any hydrogel composition known to one skilled in the art is encompassed within the invention, e.g., any of the hydrogel compositions disclosed in the following reviews: Graham, 1998, Med. Device Technol. 9 (1): 18-22; Peppas et al., 2000, Eur. J. Pharm. Biopharm. 50 (1): 27-46; Nguyen et al., 2002, Biomaterials, 23 (22): 4307-14; Henincl et al., 2002, Adv. Drug Deliv. Rev 54 (1): 13-36; Skelhorne et al., 2002, Med. Device. Technol. 13 (9): 19-23; Schmedlen et al., 2002, Biomaterials 23: 4325-32. In a specific embodiment, the hydrogel composition is applied on the umbilical cord biomaterial or membrane, that is, is disposed on the surface of the biomaterial or membrane. The hydrogel composition for example, may be sprayed onto the umbilical cord biomaterial or membrane or coated onto the surface of the biomaterial or membrane, or the biomaterial or membrane may, for example, be soaked, bathed or saturated with the hydrogel composition. In another specific embodiment, the hydrogel is sandwiched between two or more layers of umbilical cord biomaterial, or between two layers of umbilical cord membrane, or between a layer of umbilical cord membrane and biomaterial. In an even more specific embodiment, the hydrogel is sandwiched between two layers of umbilical cord biomaterial, wherein the edges of the two layers of biomaterial are sealed so as to substantially or completely contain the hydrogel.

The hydrogels useful in the umbilical cord biomaterial to be used in the ocular surgical methods of the invention can be made from any water-interactive, or water soluble polymer known in the art, including but not limited to, polyvinylalcohol (PVA), polyhydroxyehthyl methacrylate, polyethylene glycol, polyvinyl pyrrolidone, hyaluronic acid, alginate, collagen, gelatin, dextran or derivatives and analogs thereof.

In some embodiments, a composition used in an ocular surgery according to the present invention comprises an umbilical cord biomaterial or membrane, one or more bioactive compounds and a hydrogel. In other embodiments, such a composition comprises an umbilical cord biomaterial or membrane, and a hydrogel composition that comprises one or more bioactive compounds. In yet another embodiment, such a composition comprises an umbilical cord biomaterial or membrane comprising one or more bioactive compounds and a hydrogel composition comprising one or more bioactive compounds. The bioactive compounds can be, for example, one or more compounds as described in Section 5.1.2, below.

### 5.1.2. Methods of Making Umbilical Cord Biomaterial

Processed umbilical cord biomaterial useful in the ocular surgical methods of the invention can be made in a number of ways. For example, the biomaterial is preferably produced by any method that preserves the biochemical and structural characteristics of the membrane's components, chiefly collagen, elastin, laminin, and fibronectin. That is, the biomaterial can be made so as to preserve, or substantially preserve, the native structure of the protein components of the membrane or umbilical cord from which the biomaterial is made. The biomaterial may also be altered, e.g., the proteins of the biomaterial can be crosslinked, so as to improve the strength (e.g., tensile strength) of the biomaterial. The biomaterial can be completely, or substantially completely, decellularized prior to use, that is, can comprise only, or substantially only, an umbilical cord membrane, or can be made to retain other components of the umbilical cord (e.g., Wharton's jelly, umbilical vessel(s), umbilical cord cells, and the like). Generally, an umbilical cord is separated from a placenta obtained by normal birth. The umbilical cord is then cleaned and disinfected, and optionally stored for further processing, e.g., decellularization and/or drying.

In one embodiment, the umbilical cord is separated from the placenta as soon as possible after delivery of the newborn. The umbilical cord may be used immediately, or may be stored for 2-5 days from the time of delivery prior to any further treatment. Preferably, the expectant mother is screened prior to the time of birth, using standard techniques known to one skilled in the art, for communicable diseases including but not limited to, HIV, HBV, HCV, HTLV, syphilis, CMV, and other viral pathogens known to contaminate umbilical cord tissue. One exemplary method for preparing the umbilical cord biomaterial of the invention comprises the following steps:

The umbilical cord is separated from the placental disc, and is typically massaged to remove umbilical cord blood. Optionally, the umbilical cord is sectioned into pieces of about 10 cm to about 15 cm in length. The umbilical cord or umbilical cord sections can then be stored for up to about 72 hours in a sterile, preferably buffered, saline solution, such as 0.9% sterile NaCl solution. Preferably, the umbilical cord is stored under refrigeration, at a temperature of about 1° C. to about 5° C.

At this time, the umbilical cord can be slit or cut longitudinally using, e.g., a scalpel and forceps, grooved director, or the like. This allows the umbilical cord membrane to be laid flat, allowing, e.g., removal of the Wharton's jelly, and/or one or more of the umbilical cord arteries, e.g., with a forceps. The umbilical cord membrane can also be processed further without cutting and opening the membrane. An umbilical cord vessel, for example, can be removed from the cord by grasping the vessels with a forceps and gently pulling and massaging until the vessel is removed, leaving the umbilical cord membrane as an intact tube. In a preferred embodiment of deveining, the umbilical vein of a fresh (less than 48 hours after delivery) umbilical cord is canalized using the blunt probe of a vein stripper. The blunt probe is replaced with a small bullet probe, and the vein is tied to the probe with thread. The stripper is then removed, and the process is repeated with the umbilical arteries.

The umbilical cord can be further processed “as is”, wherein the cord comprises the umbilical cord membrane, vessels, and Wharton's jelly.

Continuing the embodiment, the umbilical cord or membrane can be substantially decellularized, in one embodiment a step in making umbilical cord biomaterial; that is, substantially all cellular material and cellular debris (e.g., all visible cellular material and cellular debris) can removed from the cord or membrane. Any decellularizing process known to one skilled in the art may be used, however, preferably the process used for decellularizing the umbilical cord or umbilical cord membrane does not disrupt the native conformation of the proteins making up the biomaterial. “Substantially decellularized,” as used herein, means removal of at least 90% of the cells, more preferably at least 95% of the cells, and most preferably at least 99% of the cells associated with the umbilical cord membrane. Decellularization can leave cellular material on the membrane; for example, decellularization can leave nuclear material detectable by 4′,6-diamidino-2-phenylindole (DAPI).

Decellularization can comprise physical scraping, for example, with a sterile cell scraper, in combination with rinsing with a sterile solution. The decellularization technique employed preferably does not result in gross disruption of the anatomy of the umbilical cord membrane or alter the biomechanical properties of the umbilical cord membrane.

The decellularization of the umbilical cord membrane can comprise contacting the membrane with a detergent-containing solution, such as one or more mild anionic or nonionic detergents, e.g., Triton X-100, sodium dodecyl sulfate, or the like, in an amount and for a time sufficient to decellularize the biomaterial. Any mild detergent, i.e., a non-caustic, low-foaming detergent, with a pH of about 6 to about 8, can be used to decellularize the umbilical cord biomaterial. In a specific embodiment, the biomaterial is contacted with about 0.01-1% deoxycholic acid (e.g., deoxycholic acid sodium salt monohydrate) for about 30 minutes to about 480 hours, preferably about 1 hour to about 240 hours, to decellularize the umbilical cord biomaterial. In a preferred embodiment, the umbilical cord biomaterial is decellularized in about 1% for about 20 days without scraping, followed by heat drying.

The membrane can be decellularized by any other method known to those in the art, including freezing to form intracellular ice (including, e.g., vapor phase freezing). Where freezing is used to decellularize the membrane, preferably a cryoprotectant is used, e.g., polyvinylpyrollidone at, e.g., 10% w/v, or dialyzed hydroxyethyl starch at, e.g., 10% w/v, added to standard cryopreservation solutions such as, in a non-limiting example, DMEM comprising 10% DMSO and 10% placental bovine serum.

Preferably, any native or exogenous protease activity is inhibited or prevented in the preparation of the biomaterial. Additives to the decellularization, rinse and/or storage solutions such as metal ion chelators, for example 1,10-phenanthroline and ethylenediaminetetraacetic acid (EDTA), create an environment unfavorable to many proteolytic enzymes. Providing sub-optimal conditions for proteases (e.g., collagenase) assists in protecting umbilical cord biomaterial components such as collagen from degradation during the cell lysis step. Suboptimal conditions for proteases may be achieved by formulating the decellularization solution to eliminate or limit the amount of available calcium and zinc ions. Many proteases are active in the presence of calcium and zinc ions and lose much of their activity in calcium and zinc ion free environments. Preferably, the decellularization solution is prepared, in part, by selecting conditions of pH, reduced availability of calcium and zinc ions, presence of metal ion chelators and the use of proteolytic inhibitors specific for collagenase, such that the solution will optimally lyse the native umbilical cord cells while protecting the umbilical cord biomaterial from proteolytic degradation. For example, a decellularization solution can include a buffered solution of water, pH 5.5 to 8, preferably pH 7 to 8, free from calcium and zinc ions and including a metal ion chelator such as EDTA. Decellularization can take place at, e.g., between 0° C. and 25° C., preferably below about 10° C., to reduce protease activity.

It is preferred that the decellularization treatment also limits the generation of new immunological sites. Since enzymatic degradation of, e.g., collagen is believed to lead to heightened immunogenicity, the invention encompasses treatment of the umbilical cord biomaterial with enzymes, e.g., nucleases, that are effective in inhibiting cellular metabolism, protein production and cell division, that minimize proteolysis of the components of the umbilical cord biomaterial thus preserving the underlying architecture of the amniotic biomaterial. Examples of nucleases that can be used in accordance with the methods of the invention are those effective in digestion of native cell DNA and RNA including both exonucleases and endonucleases. A non-limiting example of nucleases that can be used in accordance with the methods of the invention include exonucleases that inhibit cellular activity, e.g., DNase I (SIGMA Chemical Company, St. Louis, Mo.) and RNase A (SIGMA Chemical Company, St. Louis, Mo.) and endonucleases that inhibit cellular activity, e.g., EcoRI (SIGMA Chemical Company, St. Louis, Mo.) and HindIII (SIGMA Chemical Company, St. Louis, Mo.). It is preferable that the selected nucleases are applied in a physiological buffer solution which contains ions, e.g., magnesium, calcium, which are optimal for the activity of the nuclease. Preferably, the ionic concentration of the buffered solution, the treatment temperature and the length of treatment are selected by one skilled in the art by routine experimentation to assure the desired level of nuclease activity. The buffer is preferably hypotonic to promote access of the nucleases to cell interiors.

In another embodiment of the invention, the umbilical cord biomaterial is not decellularized prior to drying.

In another embodiment of the above steps, the umbilical cord, after initial processing, is briefly rinsed in saline to remove blood from the umbilical cord surface. The umbilical cord is then immersed in a cold deoxycholic acid solution at a concentration of about 0.1% to about 10%, and, in a specific embodiment, about 0.1% to about 2.0%. The umbilical cord is then incubated in this solution at between about 1° C. to about 8° C. for about 5 days to about 6 months. In specific embodiments, the umbilical cord is immersed, for example, for about 5 to about 15 days; about 5 to about 30 days, about 5 to about 60 days, or for up to about one year. Typically, the deoxycholic acid solution is replaced during incubation every 2-5 days. In another specific embodiment, the umbilical cord is immersed in a deoxycholic acid solution at a concentration of about 1% at a temperature of 0° C. to about 8° C. for about 5 days to about 15 days. This incubation serves two purposes. First, it allows time for serological tests to be performed on the umbilical cord and/or umbilical cord blood, so that umbilical cords failing to meet serological criteria are not processed further. Second, the longer incubation improves the removal of epithelial cells and fibroblasts, which allows for a significant reduction in the amount of time spent decellularizing the umbilical cord membrane by physically scraping. The umbilical cord biomaterial can then be dried as described below.

Following decellularization, the umbilical cord biomaterial is generally washed to assure removal of cellular debris (e.g., cellular proteins, cellular lipids, cellular nucleic acids, extracellular debris such as extracellular soluble proteins, lipids and proteoglycans, and the like). The wash solution can be de-ionized water or an aqueous hypotonic buffer. Preferably, the umbilical cord biomaterial is gently agitated, e.g., for 15-120 minutes in the detergent, e.g., on a rocking platform, to assist in the decellularization. The umbilical cord biomaterial, after detergent decellularization, can again be physically decellularized as described above; the physical and detergent decellularization steps may be repeated as necessary, as long as the integrity of the umbilical cord biomaterial is maintained, until no visible cellular material and cellular debris remain.

In certain embodiments, the umbilical cord biomaterial is dried immediately (i.e., within 30 minutes) after decellularization and/or washing. Alternatively, when further processing is not done immediately, the umbilical cord biomaterial may be refrigerated, e.g., stored at a temperature of about 1° C. to about 20° C., preferably from about 2° C. to about 8° C., for up to 28 days prior to drying. When the umbilical cord biomaterial, e.g., decellularized umbilical cord biomaterial, is stored for more than three days, the sterile solution covering the umbilical cord biomaterial is preferably changed periodically, e.g., every 1-3 days.

In certain embodiments, when the umbilical cord biomaterial is not refrigerated after washing, the biomaterial can be washed, e.g., washed at least 3 times, prior to proceeding to the next step of the preparation. In other embodiments, when the umbilical cord biomaterial has been refrigerated and the sterile solution has been changed once, the umbilical cord biomaterial can be washed at least twice prior to the next step of the preparation. In yet other embodiments, when the umbilical cord biomaterial has been refrigerated and the sterile solution has been changed twice or more, the umbilical cord biomaterial can be washed at least once prior to proceeding to the next step.

The final step in this embodiment comprises drying the decellularized umbilical cord membrane to produce the umbilical cord biomaterial of the invention. Any method of drying the umbilical cord membrane can be used. For example, the membrane can be dried using heat, one or more hygroscopic compounds, freeze-drying, vacuum, microwaving, simple evaporation, and the like, or combinations of these methods. Preferably, the biomaterial is dried under vacuum.

The umbilical cord biomaterial can be dried in any useful conformation. Preferably, the umbilical cord biomaterial is dried so as to produce a flat, dry sheet. The biomaterial can also be dried as a tube, strip, spiral, string or rope, or the like. For three-dimensional shapes, the biomaterial can be placed onto, or into, a form and dried, so that the dried biomaterial assumes the shape of the form or a part thereof. In a specific embodiment, for example, the umbilical cord membrane can be supported by rubber hose or tubing inserted from one end, and freeze-dried to form a dried tube of the umbilical cord biomaterial. At this point, the umbilical cord biomaterial can be, e.g., part of a complete umbilical cord that has been washed and rinsed; an umbilical cord biomaterial comprising Wharton's jelly but lacking vessels, an umbilical cord biomaterial that has had the interior components of the umbilical cord removed, and has been decellularized, etc. In each case, the biomaterial can be dried.

In a specific embodiment, an exemplary method for drying the umbilical cord biomaterial comprises the following steps:

Assembly of the umbilical cord biomaterial for drying. The umbilical cord biomaterial is removed from the sterile solution, and the excess fluid is gently squeezed out. The umbilical cord biomaterial is then gently stretched until it is flat with the epithelial side facing in a downward position, e.g., on a tray. The umbilical cord biomaterial is then placed on a drying frame, preferably a plastic mesh drying frame (e.g., QUICK COUNT® Plastic Canvas, Uniek, Inc., Waunakee, Wis.). In other embodiments, the drying frame may be any autoclavable material, including but not limited to a stainless steel mesh. Once the umbilical cord biomaterial is positioned on the drying frame, a sterile gauze can be placed on the drying platform of a heat dryer (or gel-dryer) (e.g., Model 583, Bio-Rad Laboratories, Hercules, Calif.), so that an area slightly larger than the umbilical cord biomaterial resting on the plastic mesh drying frame is covered. Preferably, the total thickness of the gauze layer does not exceed the thickness of one folded 4×4 gauze. Any heat drying apparatus may be used that is suitable for drying sheet-like material. The drying frame is placed on top of the gauze on the drying platform so that the edges of the plastic frame extend above beyond the gauze edges, preferably between 0.1-1.0 cm, more preferably 0.5-1.0 cm. In some embodiments, another plastic framing mesh is placed on top of the umbilical cord biomaterial. In another embodiments, a sheet of thin plastic (e.g., SW 182, clear PVC, AEP Industries Inc., South Hackensack, N.J.) or a biocompatible silicone is placed on top of the biomaterial covered mesh so that the sheet extends well beyond all of the edges. In this embodiment, the second mesh frame is not needed.

In an alternative embodiment, the umbilical cord biomaterial is placed one or more sterile sheets of TYVEK® material (e.g., a sheet of TYVEK® for medical packaging, DuPont TYVEK®, Wilmington, Del.), optionally, with one sheet of TYVEK® on top of the biomaterial (prior to placing the plastic film). This alternate process will produce a smoother version of the biomaterial (i.e., without the pattern of differential fiber compression regions along and perpendicular to the axis of the material), which may be advantageous for certain applications, such as for example for use as a matrix for expansion of cells.

Drying the umbilical cord biomaterial. In a preferred embodiment, the invention encompasses heat drying the umbilical cord biomaterial of the invention under vacuum. While the drying under vacuum may be accomplished at any temperature from about 0° C. to about 60° C., the umbilical cord biomaterial is preferably dried at between about 35° C. and about 50° C., and most preferably at about 50° C. It should be noted that some degradation of the collagen is to be expected at temperatures above 50° C. The drying temperature is preferably set and verified using a calibrated digital thermometer using an extended probe. Any amount of vacuum that can be conveniently generated can be used, but preferably, the vacuum pressure is set to about −22 inches of Hg. The drying step is continued until the umbilical cord biomaterial is substantially dry, that is, contains less than 20% water by weight, and preferably, about 3-12% water by weight as determined for example by a moisture analyzer. To accomplish this, the umbilical cord biomaterial may be heat-vacuum dried, e.g., for approximately 60 minutes to achieve a dehydrated umbilical cord biomaterial. In some embodiments, the umbilical cord biomaterial is dried for about 30 minutes to 2 hours, preferably about 60 minutes. Although not intending to be bound by any mechanism of action, it is believed that low (e.g., <50° C.) heat coupled with vacuum pressure allows the umbilical cord biomaterial to achieve the dehydrated state without denaturing collagen in the biomaterial. After completion of the drying process in accordance with the invention, the umbilical cord biomaterial can be cooled down, e.g., for approximately two minutes, with the vacuum pump running.

Packaging and Storing of the Umbilical Cord Biomaterial. Once the umbilical cord biomaterial is dried, the biomaterial is gently lifted off the drying frame. Preferably, handling of the umbilical cord biomaterial at this stage is done with sterile gloves. The umbilical cord biomaterial can be placed in a sterile container, e.g., peel pouch. When dried, the umbilical cord biomaterial produced in accordance with the methods of the invention may be stored at room temperature for an extended period of time as described supra.

In another embodiment, the umbilical cord biomaterial is prepared as above, but is not decellularized. That is, the umbilical cord membrane is obtained and dried, but the cells associated with the umbilical cord membrane are not removed. The final, dried product thus comprises, e.g., the umbilical cord membrane and/or umbilical cord vessel(s), as well as cellular components.

The umbilical cord biomaterial can be dehydrated by other methods in place of, or in addition to, the vacuum-drying method outlined above. For example, in one embodiment, the biomaterial can be freeze dried. Typically, umbilical cord biomaterial can be frozen at a temperature between about −170° C. and about 0° C. for time sufficient for the biomaterial to completely freeze. The frozen biomaterial is freeze-dried process during which the ice crystals will be removed or avoided by sublimation under vacuum.

In another embodiment, the biomaterial can be dehydrated using a solvent. For example, umbilical cord biomaterial can be dehydrated using, e.g., ethanol and acetone. In this specific embodiment, the biomaterial can be, e.g., soaked for a time in a series of ethanol-acetone mixtures (e.g., 20%, 40%, 60%, 80% and 100% ethanol, or a similar progression of equivalent solvents that act to extract water) such that the water inside the biomaterial is gradually replaced by the organic solvent. After the final soak, the biomaterial can be placed in a well-ventilated place at room temperature (about 23° C.) for a time sufficient for the solvent to evaporate. The biomaterial can alternately be vacuum-dried after the solvent soak.

In another embodiment, the biomaterial can also be dehydrated by freeze drying. For example, in a specific embodiment in a combination of the above two processes, the processed membrane can be first frozen and then transferred to a water miscible organic solvent. Ice crystals inside the membrane tissue may then be dissolved and replaced by the organic solvent using a series of progressive solvent soaks as described above. After the final soak, the biomaterial can be placed in a well-ventilated place at room temperature (e.g., about 20° C. to about 25° C.) for a time sufficient for the ethanol to evaporate. The biomaterial can alternately be vacuum-dried after the 100% ethanol soak.

Non-heat drying processes may be preferred if the porous structure of the biomaterial and/or bioactivity of the biological substances within the umbilical cord membrane need to be preserved, for example, if the biomaterial is to be used as a substrate or matrix for the transport of stem cells to a graft site, or if, e.g., the biomaterial is to be preloaded with a heat-sensitive drug as a drug release device, or if, e.g., the biomaterial is preloaded with a heat sensitive drug as a drug release device.

When the above steps are complete, the membrane generally primarily comprises collagen (types I, III, IV, V, VI and VII), glycosaminoglycans (particularly hyaluronic acid); and growth factors, particularly fibroblast growth factor (FGF), basic fibroblast growth factor (b-FGF), epidermal growth factor (EGF), insulin-like growth factor I (IGF-I), platelet-derived growth factor (PDGF) and transforming growth factor beta (TGF-β).

### 5.1.3. Other Biomaterials

The invention further encompasses the use of placental biomaterials in ocular surgery, including amniotic membrane, either fresh or dried, extracellular matrix made from placental or umbilical cord cells, and the like. Methods of making dried amniotic membrane biomaterial are generally the same as for the preparation of the dried umbilical cord biomaterial, described herein, and are described in, e.g., U.S. Patent Application Publication No. 2004/0048796, the contents of which are incorporated by reference herein in their entirety.

### 5.2. Bioactive Compounds

The placental biomaterial or umbilical cord biomaterial used in the ocular surgical methods of the invention, can comprise (e.g., be impregnated with or coated with) one or more bioactive or medicinal compounds, such as small organic molecules (e.g., drugs), antibiotics, antiviral agents, antimicrobial agents, anti-inflammatory agents, antiproliferative agents, cytokines, enzyme or protein inhibitors, antihistamines, and the like. In various embodiments, the placental biomaterial or umbilical cord biomaterial comprises, e.g., is coated or impregnated with, antibiotics (such as Clindamycin, Minocycline, Doxycycline, Gentamycin), hormones, growth factors, anti-tumor agents, anti-fungal agents, anti-viral agents, pain medications (including XYLOCAINE®, Lidocaine, Procaine, Novocaine, etc.), antihistamines (e.g., diphenhydramine, BENADRYL®, etc.), anti-inflammatory agents, anti-infectives including but not limited to antibiotics, bactericidal enzymes (such as lysozome), wound healing agents (such as cytokines including but not limited to PDGF, TGF; thymosin), hyaluronic acid as a wound healing agent, wound sealants (such as fibrin with or without thrombin), cellular attractant and scaffolding reagents (such as fibronectin), and the like, or combinations of any of the foregoing, or of the foregoing and other compounds not listed. Such impregnation or coating may be accomplished by any means known in the art, and a portion or the whole of the placental biomaterial may be so coated or impregnated.

The placental biomaterial or umbilical cord biomaterial, or composite comprising the same, may comprise any of the compounds listed herein, without limitation, individually or in any combination. Any of the biologically active compounds listed herein may be formulated by known methods for immediate release or extended release. Additionally, the biomaterial may comprise two or more biologically active compounds in different manners; e.g., the biomaterial may be impregnated with one biologically active compound and coated with another. In another embodiment, the placental biomaterial or umbilical cord biomaterial comprises one biologically active compound formulated for extended release, and a second biologically active compound formulated for immediate release.

Wound healing requires adequate nutrition, particularly the presence of iron, zinc, vitamin C, arginine, and the like. Thus, the placental biomaterial or umbilical cord biomaterial, or composite comprising the same, may comprise, e.g., be impregnated or coated with, a physiologically-available form of one or more nutrients required for wound healing. Preferably, the nutrient is formulated for extended release.

The placental biomaterial or umbilical cord biomaterial, or composite comprising the same, may comprise an antibiotic. In certain embodiments, the antibiotic is a macrolide (e.g., tobramycin (TOBI®)), a cephalosporin (e.g., cephalexin (KEFLEX®)), cephradine (VELOSEF®)), cefuroxime (CEFTIN®, cefprozil (CEFZIL®), cefaclor (CECLOR®), cefixime (SUPRAX® or cefadroxil (DURICEF®), a clarithromycin (e.g., clarithromycin (Biaxin)), an erythromycin (e.g., erythromycin (EMYCIN®)), a penicillin (e.g., penicillin V (V-CILLINK® or PEN VEEK®)) or a quinolone (e.g., ofloxacin (FLOXIN®), ciprofloxacin (CIPRO®) ornorfloxacin (NOROXIN®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), macrolides (e.g., azithromycin, carbomycin, clarithomycin, dirithromycin, erythromycin, and erythromycin acistrate), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, ciprofloxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

The placental biomaterial or umbilical cord biomaterial, or a composite comprising the same, may comprise, e.g., be coated or impregnated with, an antifungal agent. Suitable antifungal agents include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofuldin.

The placental biomaterial or umbilical cord biomaterial, or a composite comprising the same, may comprise, e.g., be coated or impregnated with, an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; and other anti-inflammatory agents including, but not limited to, methotrexate, colchicine, allopurinol, probenecid, sulfinpyrazone and benzbromarone.

The placental biomaterial or umbilical cord biomaterial, or a composite comprising the same, may comprise, e.g., be coated or impregnated with, an antiviral agent. Useful antiviral agents include, but are not limited to, nucleoside analogs, such as zidovudine, acyclovir, gangcyclovir, vidarabine, idoxuridine, trifluridine, and ribavirin, as well as foscarnet, amantadine, rimantadine, saquinavir, indinavir, ritonavir, and the alpha-interferons.

The placental biomaterial or umbilical cord biomaterial, or a composite comprising the same, comprises, e.g., may be coated or impregnated with, a cytokine receptor modulator. Examples of cytokine receptor modulators include, but are not limited to, soluble cytokine receptors (e.g., the extracellular domain of a TNF-α receptor or a fragment thereof, the extracellular domain of an IL-10 receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-α, TNF-β, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-10 antibodies, anti-IL-6 antibodies, anti-IL-8 antibodies (e.g., ABX-IL-8 (Abgenix)), and anti-IL-12 antibodies). In a specific embodiment, a cytokine receptor modulator is IL-4, IL-10, or a fragment thereof. In another embodiment, a cytokine receptor modulator is an anti-IL-1 antibody, anti-IL-6 antibody, anti-IL-12 receptor antibody, or anti-TNF-α antibody. In another embodiment, a cytokine receptor modulator is the extracellular domain of a TNF-α receptor or a fragment thereof. In certain embodiments, a cytokine receptor modulator is not a TNF-α antagonist.

In a preferred embodiment, proteins, polypeptides or peptides (including antibodies) that are utilized as immunomodulatory agents are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as immunomodulatory agents are human or humanized.

The placental biomaterial or umbilical cord biomaterial, or a composite comprising the same, may also comprise, e.g., be coated or impregnated with, a cytokine. Examples of cytokines include, but are not limited to, colony stimulating factor 1 (CSF-1), interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), insulin-like growth factor 1 (IGF-1), platelet derived growth factor (PDGF), erythropoietin (Epo), epidermal growth factor (EGF), fibroblast growth factor (FGF) (basic or acidic), granulocyte macrophage stimulating factor (GM-CSF), granulocyte colony stimulating factor (G-CSF), heparin binding epidermal growth factor (HEGF), macrophage colony stimulating factor (M-CSF), prolactin, and interferon (IFN), e.g., IFN-alpha, and IFN-gamma), transforming growth factor alpha (TGF-α), TGFβ1, TGFβ2, tumor necrosis factor alpha (TNF-α), vascular endothelial growth factor (VEGF), hepatocyte growth factor (HGF), etc.

The placental biomaterial or umbilical cord biomaterial, or composite comprising the same, may also comprise, e.g., be coated or impregnated with, a hormone. Examples of hormones include, but are not limited to, luteinizing hormone releasing hormone (LHRH), growth hormone (GH), growth hormone releasing hormone, ACTH, somatostatin, somatotropin, somatomedin, parathyroid hormone, hypothalamic releasing factors, insulin, glucagon, enkephalins, vasopressin, calcitonin, heparin, low molecular weight heparins, heparinoids, synthetic and natural opioids, insulin thyroid stimulating hormones, and endorphins. Examples of β-interferons include, but are not limited to, interferon β 1-a and interferon β 1-b.

The placental biomaterial or umbilical cord biomaterial, or composite comprising the same, may also comprise, e.g., be coated or impregnated with, an alkylating agent. Examples of alkylating agents include, but are not limited to nitrogen mustards, ethylenimines, methylmelamines, alkyl sulfonates, nitrosoureas, triazenes, mechlorethamine, cyclophosphamide, ifosfamide, melphalan, chlorambucil, hexamethylmelaine, thiotepa, busulfan, carmustine, streptozocin, dacarbazine and temozolomide.

The placental biomaterial or umbilical cord biomaterial, or a composite comprising the same, may also comprise, e.g., be coated or impregnated with, an immunomodulatory agent, including but not limited to methotrexate, mitomycin C, leflunomide, cyclophosphamide, cyclosporine A, macrolide antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, and cytokine receptor modulators. peptide mimetics, and antibodies (e.g., human, humanized, chimeric, monoclonal, polyclonal, Fvs, ScFvs, Fab or F(ab)₂ fragments or epitope binding fragments), nucleic acid molecules (e.g., antisense nucleic acid molecules and triple helices), small molecules, organic compounds, and inorganic compounds. In particular, immunomodulatory agents include, but are not limited to, methothrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, and cytokine receptor modulators. Examples of T cell receptor modulators include, but are not limited to, anti-T cell receptor antibodies (e.g., anti-CD4 antibodies (e.g., cM-T412 (Boehringer), IDEC-CE9.Is (IDEC and SKB), mAb 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies, anti-CD1 1a antibodies (e.g., Xanelim (Genentech)), and anti-B7 antibodies (e.g., IDEC-114) (IDEC))) and CTLA4-immunoglobulin. In a specific embodiment, a T cell receptor modulator is a CD2 antagonist. In other embodiments, a T cell receptor modulator is not a CD2 antagonist. In another specific embodiment, a T cell receptor modulator is a CD2 binding molecule, preferably MEDI-507. In other embodiments, a T cell receptor modulator is not a CD2 binding molecule.

The amount of the bioactive compound coating or impregnating the placental biomaterial or umbilical cord biomaterial, or composite comprising the same, may vary, and will preferably depend upon the particular bioactive compound to be delivered, and the effect desired. For example, where the bioactive compound is an anti-inflammatory agent, the amount of the anti-inflammatory agent on or contained by the placental biomaterial is an amount sufficient to measurably reduce one or more symptoms or indicia of inflammation in a tissue contacted by, or proximal to, e.g., an placental biomaterial implant.

In various embodiments, the placental biomaterial or umbilical cord biomaterial, or composite comprising the same, may comprise, e.g., be coated or impregnated with, at least 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 100, 1250, 1500, 2000, 2500, 300, 3500, 4000, 4500, 5000, 5500, 6000, 6500, 7000, 7500, 8000, 8500, 9000, 9500, 10000, 20000, 30000, 40000, 50000, 60000, 70000, 80000, 90000, 100000, 200000, 300000, 400000, 500000, 600000, 700000, 800000, 900000 or at least 1000000 nanograms of a bioactive compound. In another embodiment, the placental biomaterial or umbilical cord biomaterial, or composite comprising the same, may be coated with, or impregnated with, no more than 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 100, 1250, 1500, 2000, 2500, 300, 3500, 4000, 4500, 5000, 5500, 6000, 6500, 7000, 7500, 8000, 8500, 9000, 9500, 10000, 20000, 30000, 40000, 50000, 60000, 70000, 80000, 90000, 100000, 200000, 300000, 400000, 500000, 600000, 700000, 800000, 900000 or at least 1000000 nanograms of a bioactive compound.

### 5.3. Conformation of the Placental Biomaterial

The placental biomaterial or umbilical cord biomaterial, used in the ocular surgical methods of the invention can be used in any shape or conformation that facilitates such surgery, or the treatment or repair of a discontinuity or trauma in the eye, eyelid, or surrounding tissues caused by, or created as an adjunct to, such surgery or that is the subject of such surgery. In particular, the biomaterial may be formed into any shape or conformation that will facilitate its use in the methods of the invention. For example, the biomaterial can be formed into any shape or conformation that will facilitate, e.g., treatment or healing of a discontinuity produced in, or as an adjunct to, ocular surgery, e.g., a fistula, an incision, sutures; etc. The biomaterial can be provided as square, rectangular, circular or oval shaped pieces, or may be cut to conform generally to the shape of, e.g., a cornea, eye muscle, etc.

In various embodiments, placental biomaterial or umbilical cord biomaterial is provided as pieces measuring approximately 0.25 cm×0.5 cm, 0.25 cm×0.75 cm, 0.25 cm×1 cm, 0.5×0.5 cm, 0.5 cm×0.75 cm, 0.5 cm×1 cm, 0.75 cm×0.75 cm, 0.75×1 cm, 1×1 cm, 1.5×1.5 cm, 2×2 cm, 2.5×2.5 cm, 1×1.5 cm, 1×2 cm, 1×2.5 cm, 1×3 cm, 1×3.5 cm, 1×4 cm, 1×4.5 cm, 1×5 cm, 1×5.5 cm, 1×6 cm, 1×6.5 cm, 1×7 cm, 1.5×2 cm, 1.5×2.5 cm, 1.5×3 cm, 1.5×3.5 cm, 1.5×4 cm, 1.5×4.5 cm, 1.5×5 cm, 1.5×5.5 cm, 1.5×6 cm, 1.5×6.5 cm, 1.5×7 cm, 2×2.5 cm, 2×3 cm, 2×3.5 cm, 2×4 cm, 2×4.5 cm, 2×5 cm, 2×5.5 cm, 2×6 cm, 2×6.5 cm, 2×7 cm, 2.5×2.5 cm, 2.5×3 cm, 2.5×3.5 cm, 2.5×4 cm, 2.5×4.5 cm, 2.5×5 cm, 2.5×5.5 cm, 2.5×6 cm, 2.5×6.5 cm, 2.5×7 cm, 3×3 cm in size, or may be no smaller, or no larger, than 0.25 cm×0.5 cm, 0.25 cm×0.75 cm, 0.25 cm×1 cm, 0.5×0.5 cm, 0.5 cm×0.75 cm, 0.5 cm×1 cm, 0.75 cm×0.75 cm, 0.75×1 cm, 1×1 cm, 1.5×1.5 cm, 2×2 cm, 2.5×2.5 cm, 1×1.5 cm, 1×2 cm, 1×2.5 cm, 1×3 cm, 1×3.5 cm, 1×4 cm, 1×4.5 cm, 1×5 cm, 1×5.5 cm, 1×6 cm, 1×6.5 cm, 1×7 cm, 1.5×2 cm, 1.5×2.5 cm, 1.5×3 cm, 1.5×3.5 cm, 1.5×4 cm, 1.5×4.5 cm, 1.5×5 cm, 1.5×5.5 cm, 1.5×6 cm, 1.5×6.5 cm, 1.5×7 cm, 2×2.5 cm, 2×3 cm, 2×3.5 cm, 2×4 cm, 2×4.5 cm, 2×5 cm, 2×5.5 cm, 2×6 cm, 2×6.5 cm, 2×7 cm, 2.5×2.5 cm, 2.5×3 cm, 2.5×3.5 cm, 2.5×4 cm, 2.5×4.5 cm, 2.5×5 cm, 2.5×5.5 cm, 2.5×6 cm, 2.5×6.5 cm, 2.5×7 cm, 3×3 cm, though the biomaterial may be cut to different dimensions. Longer and/or wider pieces can be formed by laminating two or more smaller pieces, as described elsewhere herein. Further, the biomaterial may be provided as a sheet from which an end user may cut two or more pieces, or may be provided as a roll or strip.

The placental biomaterial or umbilical cord biomaterial may be provided to the end user fresh (e.g., not dried), dry, or pre-wetted in a suitable physiologically-compatible, medically-useful liquid, such as a saline solution. In one embodiment, the solution comprises one or more bioactive compounds, as described in Section 5.1.2, above.

### 5.4. Storage and Handling of Placental Biomaterial

Placental biomaterial or umbilical cord biomaterial can be stored in any manner that facilitates its use in ocular surgery.

Processed placental biomaterial or umbilical cord biomaterial, e.g., dehydrated umbilical cord membrane or dehydrated amniotic membrane, may be stored, e.g., as dehydrated sheets, at room temperature (e.g., 25° C.) prior to use. In certain embodiments, the biomaterial can be stored at a temperature of at least 10° C., at least 15° C., at least 20° C., at least 25° C., or at least 29° C. Preferably, placental biomaterial or umbilical cord biomaterial, in dehydrated form, is not refrigerated. In some embodiments, the biomaterial may be refrigerated at a temperature of about 2° C. to about 8° C. Dried biomaterial can be stored at any of the specified temperatures for 12 months or more with no alteration in biochemical or structural integrity (e.g., no degradation), without any alteration of the biochemical or biophysical properties of the umbilical cord biomaterial. Dried biomaterial can be stored for several years with no alteration in biochemical or structural integrity (e.g., no degradation), without any alteration of the biochemical or biophysical properties of the biomaterial. The biomaterial can be stored in any container suitable for long-term storage. Preferably, the biomaterial used in the methods of the invention is stored in a sterile double peel-pouch package.

The placental biomaterial or umbilical cord biomaterial, in embodiments in which the material has been dried, may be hydrated prior to use, using, e.g., a sterile physiological buffer. In a specific embodiment, the sterile saline solution is a 0.9% NaCl solution. In some embodiments the sterile saline solution is buffered. In certain embodiments, the hydration of the biomaterial requires at least 2 minutes, at least 5 minutes, at least 10 minutes, at least 15 minutes, or at least 20 minutes. In a preferred embodiment, the hydration of the biomaterial is complete within 5 minutes. In yet another preferred embodiment, the hydration of the biomaterial of the invention is complete within 10 minutes. In yet another embodiment, the hydration of the biomaterial takes no more than 10 minutes. Once hydrated, the biomaterial can be maintained in solution, e.g., in sterile 0.9% NaCl solution, for up to six months, with a change of solution, e.g., every three days.

### 5.4.1. Sterilization

Sterilization of placental biomaterial or umbilical cord biomaterial, may be accomplished by any medically-appropriate means, preferably means that do not significantly alter the tertiary and quaternary structure of the biomaterial or membrane proteins. Sterilization can be accomplished, for example, using gas, e.g., ethylene dioxide. Sterilization can also be accomplished using radiation, for example, gamma radiation, and is preferably done by electron beam irradiation using methods known to one skilled in the art, e.g., Gorham, D. Byrom (ed.), 1991, Biomaterials, Stockton Press, New York, 55-122. Use of any dose of radiation sufficient to kill at least 99.9% of bacteria or other potentially contaminating organisms is within the scope of the invention. In a preferred embodiment, a dose of at least 18-25 kGy is used to achieve the terminal sterilization of the placental biomaterial.

### 5.4.2. Laminates

Placental biomaterial or umbilical cord biomaterial, useful in the ocular surgical methods of the invention, may be laminated to provide, e.g., greater suturability. Laminates of the biomaterial can comprise biomaterial, e.g., from a single amniotic membrane or umbilical cord, or piece thereof, wherein the biomaterial is folded once, or a plurality of times, longitudinally or laterally, or both. Laminates of the placental biomaterial can also comprise two or more sheets of placental biomaterial.

In a specific embodiment, the biomaterial is umbilical cord biomaterial. Umbilical cord biomaterial, being anisotropic, has two orientations, longitudinal (that is, along the length of the umbilical cord membrane) and lateral (that is, around the width of the umbilical cord). Where a laminate comprises two sheets of the umbilical cord biomaterial, the sheets can be laminated so that each sheet is oriented the same way (e.g., each sheet oriented longitudinally), or such that at least one sheet is oriented laterally and one longitudinally. In other embodiments, each of the layers of umbilical cord biomaterial can be laminated in any orientation with respect to any other layer of the biomaterial in the laminate.

Additionally, the umbilical cord has a sidedness; that is, the umbilical cord biomaterial has an epithelial side (that is, the side towards the interior of the umbilical cord) and a mesothelial side (that is, the side towards the exterior of the umbilical cord). Laminates can comprise two or more layers of the umbilical cord biomaterial in any sidedness configuration. For example, laminates can comprise layers of umbilical cord biomaterial in which only the endothelial sides of the layers are in contact; only the mesothelial sides of the layers are intact; or a combination of both. In one embodiment, a laminate comprises four layers, wherein two sets of two layers, contacted endothelial to mesothelial sides, are contacted by the exposed mesothelial side such that the two faces of the laminate show the endothelial sides.

Placental biomaterial or umbilical cord biomaterial can be laminated, e.g., by folding a single sheet of biomaterial, or by stacking 2 or more layers of the biomaterial one atop the other, and sealing or drying. The biomaterial may be laminated either dry or after rehydration. Alternatively, two or more layers of, e.g., amniotic membrane or umbilical cord biomaterial, or composition comprising an amniotic membrane or umbilical cord biomaterial, can be laminated during processing prior to initial drying after cell removal, e.g., after a cell scraping step. If laminated prior to the initial drying, 2 or more biomaterial layers can be stacked one atop the other and subsequently dried, using, for example, a freeze-drying process, or drying under moderate heat with or without vacuum. The heat applied preferably is not so intense as to cause breakdown or decomposition of the protein components, especially the collagen, of the biomaterial. Typically, the heat applied is less than about 70° C., preferably less than about 60° C., and, more preferably, is approximately 50° C. Lamination time varies with, e.g., the number of layers being laminated, but typically takes 1-2 hours at 50° C.

Placental biomaterial or umbilical cord biomaterial may also be laminated using an adhesive applied between 2 or more layers of biomaterial. Such an adhesive is preferably appropriate for medical applications, and can comprise a natural biological adhesive, for example fibrin glue, a synthetic adhesive, or combinations thereof. The adhesive may further be chemically converted from precursors during the lamination process.

Laminates of the placental biomaterial or umbilical cord biomaterial can comprise, for example, biomaterial that has been decellularized, biomaterial that retains the cellular material (that is, where the cells have been killed, but not removed), or biomaterial comprising living cells, e.g., umbilical cord cells or placental cells, including stem cells, or cells of another type (e.g., where cells have been cultured on a sheet of biomaterial).

The laminates useful in the present invention can comprise only placental biomaterial, only umbilical cord biomaterial, or a combination of placental biomaterial and umbilical cord biomaterial, with or without additional components. In one embodiment, a laminate of the invention comprises one layer of umbilical cord biomaterial and one layer of placental biomaterial. In another embodiment, the laminate of the invention comprises a layer of umbilical cord biomaterial between two layers of placental biomaterial. In another embodiment, the laminate of the invention comprises a layer of amniotic membrane between two layers of umbilical cord biomaterial. In another embodiment, the laminate of the invention comprises a layer of placental biomaterial and two layers of umbilical cord biomaterial, wherein said layers of umbilical cord biomaterial are adjacent to each other. In another embodiment, the laminate of the invention comprises a layer of umbilical cord biomaterial and two layers of placental biomaterial, wherein said layers of placental biomaterial are adjacent to each other. The laminates of the invention can also comprise pluralities of layers of umbilical cord biomaterial and placental biomaterial layered in any order.

A laminate comprising placental biomaterial and/or umbilical cord biomaterial can comprise a second type of material, e.g., one or more layers of placental biomaterial, such as amniotic membrane, and/or umbilical cord biomaterial, such as umbilical cord membrane, can be layered with one or more layers of a second biologically-compatible material, e.g., a sheetlike material such as, e.g., pericardium or dura mater. Where the second material has a “grain” or orientation, the biomaterial can be laminated such that the biomaterial lies with its longitudinal direction along, or alternatively across, the grain of the second material. The biomaterial can also be laminated with a non-biological material, e.g., plastic, e.g., PROLENE®, nylon, VICRYL®, TYVEK® or the like. In a specific embodiment, the non-biological material is a mesh.

In one embodiment, a laminate useful in ocular surgery comprises at least two sheets of placental biomaterial and/or umbilical cord biomaterial approximately the same size and shape laid one atop the other so that the shape is substantially maintained. Such a laminate can be trimmed to finalize a particular shape. In another embodiment, a laminate comprises two or more sheets of biomaterial, wherein a portion of each of the sheets overlaps another. In a specific embodiment, such a laminate of overlapping sheets of the biomaterial can itself be laminated with another layer of a material, e.g., another overlapping biomaterial laminate; individual sheets of umbilical cord biomaterial; another type of biomaterial, e.g., an amniotic membrane-derived biomaterial; an artificial sheet or film; etc.

### 5.5. Stem Cells

The placental biomaterial or umbilical cord biomaterial as described herein can also comprise stem or progenitor cells. The biomaterial can comprise, e.g., mesenchymal or mesenchymal-like stem cells, for example, those described in U.S. Pat. Nos. 5,486,359, 6,261,549 and 6,387,367, or placental stem cells such as those described in U.S. Application Publication Nos. 2002/0123141, 2003/0032179 and 2003/0180269. However, the biomaterial may comprise stem or progenitor cells, preferably mammalian stem or progenitor cells, from any tissue source. The biomaterial can comprise embryonic stem cells or embryonic germ cells. In a preferred embodiment, the placental biomaterial or umbilical cord biomaterial comprises limbal stem cells, cells that can differentiate into limbal stem cells, or cells that can differentiate into limbal cells.

The placental biomaterial or umbilical cord biomaterial, and stem or progenitor cells, can be combined, e.g., in advance of a procedure in which the biomaterial is contacted with an individual having a disease, disorder or condition that would be amenable to treatment using an umbilical cord biomaterial. For example, stem cells can be contacted with, e.g., disposed onto, the biomaterial sufficiently in advance of such a procedure for a plurality, a majority, or substantially all of the stem cells to adhere to the biomaterial. The stem cells can be contacted with the biomaterial immediately before the biomaterial is contacted with the individual. The stem cells can also be contacted with the biomaterial in situ, after the biomaterial is contacted with the individual. The number of stem or progenitor cells disposed onto the surface of the umbilical cord biomaterial may vary, but may be at least about 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹, or 1×10¹²; or may be no more than 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹, or 1×10¹² stem or progenitor cells.

The stem cells, at any of the times noted above, can be contacted with one or more differentiation-modulating agents, for example, the differentiation-modulating agents described in U.S. Application Publication Nos. 2003/0235909 and/or 2004/0028660, the disclosures of which are incorporated by reverence in their entireties herein, or International Application Publication No. WO 03/087333. Methods of differentiating stem cells to, for example, epidermal, mesodermal, and other cell types are known in the art, and are described, e.g., in U.S. Application Publication No. 2004/0028660.

### 5.6. Uses of Placental Biomaterial and Umbilical Cord Biomaterial in Ocular Surgery

Placental biomaterial or umbilical cord membrane biomaterial, can be used in any ocular surgery in which a tissue patch or graft can be, or would ordinarily be, used. For example, in various embodiments, placental biomaterial or umbilical cord biomaterial can be used to patch or repair discontinuities in, or trauma to, a structure of the eye or eyelid, or in the surrounding tissues, that is caused by or is an adjunct to an ocular surgery, including, but not limited to, incisions, fistulas (e.g., needle holes, an access hole in the sclera made to allow the passage of a surgical instrument, burns, etc.) whether deliberately or accidentally made. The ocular surgery can also be a surgery to repair or ameliorate trauma to the eye. Methods of performing ocular surgeries listed herein, and similar surgeries, are well-known in the art and are readily practiced by those of skill in the art.

Thus, in one embodiment, the invention provides a method of performing an ocular surgical procedure that involves a structure of an eye or tissue adjacent to an eye, comprising contacting said structure with placental biomaterial or umbilical biomaterial. In a specific embodiment, said placental biomaterial is processed, e.g., dried, umbilical cord biomaterial. In another specific embodiment, the discontinuity is a hole or opening (fistula), and said contacting substantially or completely closes the fistula. Such a fistula can be, e.g., an incision made during said surgery, e.g., a scleral buckle. In a specific embodiment, said ocular procedure is a glaucoma surgery. Examples of glaucoma surgery are provided in Section 5.6.1, below. In another specific embodiment, said ocular procedure is a cataract surgery. Examples of cataract surgeries are provided in Section 5.6.2, below. In another specific example, said ocular surgery is a corneal surgery to, e.g., reshape the corneal surface, e.g., refractive surgery. Examples of corneal and refractive surgery are provided in Section 5.6.3, below. In another specific example, said ocular surgery is an eye muscle surgery. Examples of eye muscle surgeries are provided in Section 5.6.4, below. In another specific example, said ocular surgery is a vitreo-retinal surgery. Examples of vitreo-retinal surgeries are provided in Section 5.6.5, below. In another specific example, said ocular surgery is an oculoplastic surgery. Examples of oculoplastic surgeries are provided in Section 5.6.6, below.

Generally as appropriate, the biomaterial is held in place at an ocular surgical site by stapling, suturing, gluing with an artificial or biodegradable glue or cement, or other medically-acceptable method, or can be held in place by tissues surrounding the surgical site.

The biomaterial can be held in place, for example, by a tissue glue or tissue adhesive, e.g., a cyanoacrylate (e.g., N-butyl-2-cyanoacrylate, HISTOACRYL™), fibrin glue, fibrinogen glue, a hydrogel tissue glue, chondroitin sulfate aldehyde, natural proteins (e.g., BD Cell-Tak™ (BD Biosciences)), and the like. The biomaterial can also be held in place by a suture, e.g., resorbable or non-resorbable sutures.

### 5.6.1. Glaucoma Surgeries

Glaucoma is a group of diseases of the eye characterized by optic nerve neuropathy, particularly the loss of optic ganglion cells, which is frequently associated with raised intraocular pressure. Many glaucoma surgeries act to reduce the intraocular pressure to a pressure that is normal, or tolerable by the optic nerve.

In the most common surgical procedure to correct glaucoma, a trabeculectomy, also known as a filtration surgery, a partial thickness flap is made in the scleral wall of the eye, and a window opening made under the flap to remove a portion of the trabecular meshwork. The scleral flap is then sutured loosely back in place, allowing fluid to flow out of the intraocular space through this opening, resulting in lowered intraocular pressure. Scarring can occur around or over the flap opening, causing it to become less effective or lose effectiveness altogether.

Thus, in one aspect, the invention provides a method of treating glaucoma comprising performing a trabeculectomy in which a partial thickness flap is made in the scleral wall, wherein at least a portion of the flap is contacted with placental biomaterial or umbilical cord biomaterial. In a specific embodiment, said biomaterial comprises a hole through which intraocular fluid can pass.

In a trabeculectomy, a bleb generally forms over the flap created during the procedure. Occasionally, the bleb leaks, a complication that can cause blindness. It is desirable that a leaking bleb be repaired, e.g., by patching. Therefore, in another embodiment, the invention provides a method of repairing a leaking bleb, wherein the bleb results from an eye surgery such as a trabeculectomy, by contacting the bleb with placental biomaterial or umbilical cord biomaterial, such that leakage from said bleb is detectably reduced or is stopped.

Another complication of a trabeculectomy is the formation of a scar at the point of incision, which occurs in approximately 20% of patients undergoing the surgery. Such scarring can be prevented or reduced by contacting the incision at the point at risk for scarring with placental biomaterial or umbilical cord biomaterial.

In other surgeries to treat glaucoma, one or more small tubes are inserted into the anterior chamber of the eye and out underneath the conjunctiva to allow flow of fluid out of the eye; i.e., a silicone tube, a Molteno implant, Kupin-Denver or Bearveldt tube shunt, e.g., a trabeculectomy. Placental biomaterial or umbilical cord biomaterial, e.g., a laminate of placental biomaterial and/or umbilical cord biomaterial, can be contacted with, e.g., placed over the exposed section of the tube in order to prevent its dissolution, or delay the dissolution of the tube as compared to a tube not so contacted. Thus, in another embodiment, the invention provides a method of treating glaucoma comprising inserting a tube into the anterior chamber of the eye, said tube allowing passage of vitreous humor, wherein at least a portion of said tube is contacted with placental biomaterial or umbilical cord biomaterial such that dissolution of said tube is prevented or delayed compared with a tube not contacted with placental biomaterial or umbilical cord biomaterial. In a specific embodiment, said biomaterial is umbilical cord biomaterial, e.g., unprocessed umbilical cord membrane or dried umbilical cord membrane.

In another embodiment, the glaucoma surgery comprises the creation of a flap consisting of conjunctiva and Tenon's capsule as part of a glaucoma filtering operation. A perforation of the flap can be repaired by contacting, e.g., substantially covering the perforation with placental biomaterial, e.g., umbilical cord membrane or umbilical cord biomaterial.

In other specific embodiments, the ocular surgeries in which placental biomaterial or umbilical cord biomaterial can be used includes, but is not limited to, trebeculoplasty, iridotomy, iridactomy, a filtering surgery, anterior sclerotomy, sclerostomy, penetrating trabeculotomy, non-penetrating trabeculotomy, trepanotrabeculectomy, goniotomy, cyclotomy, cycloanemization, cyclocryotherapy, cyclocryopexy, iridenclesis, iridocyclectomy, iridosclerotomy, corenclisis or goniocurretage.

### 5.6.2. Cataract Surgeries

Cataract surgery involves removal of a cataract, an opacity that develops in the crystalline lens of the eye. Typically, the lens is surgically removed and replaced with an artificial, e.g., plastic, intraocular lens. Two main types of cataract surgeries are intracapsular cataract extraction (ICCE) and extracapsular cataract extraction (ECCE). In intracapsular cataract surgery, the lens and lens capsule are removed and replaced with an artificial lens. In estracapsular cataract surgery, the capsule surrounding the lens is opened enough to replace the existing lens with the artificial lens in the capsule. Extracapsular cataract surgery can involve a technique called phacoemulsification, in which the lens is disrupted with ultrasonic energy and removed with suction. Occasionally, surrounding tissue is disrupted in the process, resulting in phacoemulsification burns, also known as phaco burns. Conventional extracapsular cataract surgery involves removal of the lens through an incision made in the cornea or sclera.

In another aspect, therefore, the invention provides for the use in cataract surgery of placental biomaterial or umbilical cord biomaterial. For example, the biomaterial can be placed on any part of the eye, particularly the exterior of the eye, that is disrupted as part of a cataract surgery, to aid in healing. Thus, in one embodiment, the invention provides a method of cataract surgery comprising contacting the eye with placental biomaterial or umbilical cord biomaterial at one or more points in which a discontinuity is made in an eye as part of said cataract surgery, e.g., covering the discontinuity with the biomaterial. The contacting, in certain embodiments, can comprise the use of glue to hold the biomaterial in place. In specific embodiments, the discontinuity is an opening in the sclera or other part of the eye made to facilitate removal of the lens, lens capsule, or both. In another specific embodiment, said discontinuity is a burn or disruption of non-lens or non-lens capsule tissue caused during phacoemulsification.

### 5.6.3. Corneal Surgeries And Refractive Surgeries

Refractive surgeries generally involve the reshaping of the cornea of the eye to alter the path of light entering the eye and striking the retina to produce an apparently sharper visual image. Several different methodologies have emerged to accomplish this goal. See., e.g., U.S. Pat. Nos. 4,665,913; 4,648,400; 4,669,466; 4,732,148; 4,770,172; 4,773,414; 5,163,9034. In one methodology, a corneal flap is made with a microtome or laser and pulled back, and corneal tissue under the flap is removed with a laser. The flap is then replaced. This technique is used in LASIK (Laser-Assisted In Situ Keratomileusis) and LASEK (Laser-Assisted Sub-Epithelial Keratectomy). Side effects of LASIK or LASEK include corneal hazing or “starring” caused by regrowth of corneal tissue. In another methodology, an excimer laser is used to make incisions in the cornea radiating from the pupil, changing the shape of the cornea. This technique is used in PRK (Photo-Refractive Keratectomy). PRK is generally less desirable than LASIK or LASEK due to post-operative pain, corneal hazing, and a longer time to recovery of normal vision. Other, less common refractive methodologies include automated lamellar keratoplasty (ALK), laser thermal keratoplasty (LTK), and conductive keratoplasty (CK). In each case, the use of placental biomaterial, e.g., umbilical cord membrane or umbilical cord biomaterial, can improve the healing process and reduce the amount or degree of corneal hazing.

Thus, in another aspect, the invention provides a method of corneal or refractive surgery comprising contacting at least a portion of the cornea on which the surgery was performed with placental biomaterial or umbilical cord biomaterial. In a specific example, the refractive surgery is PRK. In another specific embodiment, the refractive surgery is LASIK or LASEK. In another specific embodiment, said refractive surgery is automated lamellar keratoplasty (ALK), laser thermal keratoplasty (LTK), or conductive keratoplasty (CK).

In another specific example of the method, the biomaterial is, or is part of, a bandage contact lens. A bandage contact lens is a temporary contact lens used to facilitate healing of the cornea after surgery or other injury. In the context of the present invention, a preferred bandage contact lens is one comprising or made from umbilical cord membrane, more preferably from umbilical cord biomaterial. In one embodiment, the contact lens consists of placental biomaterial or umbilical cord biomaterial. In another embodiment, the bandage contact lens comprises placental biomaterial or umbilical cord biomaterial, and a second material. The placental or umbilical cord biomaterial can be distributed on the second material as a thin membrane or as a discontinuous layer (e.g., as homogenized, powdered, etc. biomaterial). The placental or umbilical cord biomaterial can be affixed to the second material by methods known in the art, e.g., by the methods disclosed in U.S. Pat. No. 4,973,493, the contents of which are incorporated by reference herein in their entirety. In a specific embodiment of the bandage contact lens, the lens has a hole substantially in the center of the lens, adjacent to the pupil, to allow the wearer to see clearly through the lens for at least a portion of the visual field. Thus, in a specific embodiment, the invention provides a method of refractive surgery comprising performing refractive surgery on a cornea and contacting said cornea with a bandage contact lens comprising placental biomaterial or umbilical cord biomaterial. In a more specific embodiment, the biomaterial is umbilical cord biomaterial. In another more specific embodiment, said refractive surgery is PRK.

### 5.6.4. Eye Muscle Surgeries

Eye muscle surgeries are the third most common eye surgery in the U.S. Eye muscle surgeries typically correct strabismus, a condition in which the eyes do not point in the same direction. Eye muscle surgery can involve recession, the moving of the insertion of n eye muscle posteriorly towards its origin; advancement, the moving of the eye muscle from its original place of attachment to a more forward position; myectomy (the excision of a portion of an eye muscle); myotomy (the surgical division of an eye muscle); tenectomy (the resection of a tendon of an eye muscle); tenotomy (the surgical division of a tendon to correct a deformity of a muscle), and the like. Eye muscle surgeries can tighten an eye muscle, loosen an eye muscle, or reposition an eye muscle.

Thus, in another aspect, the invention provides a method of performing a surgical procedure on an eye muscle, comprising contacting the eye muscle with placental biomaterial or umbilical cord biomaterial. Generally, the method is accomplished by contacting the eye muscle with the biomaterial at the point at which the surgery is performed on the muscle, and can contact the muscle alone, or the muscle and one or more underlying tissues, e.g., epithelium, tissue lining the orbital bone of the eye socket, the orbital bone itself, etc. The biomaterial can be held in place using, e.g., staples or sutures, or can be held in place by tissues surrounding the eye muscle. As appropriate for the particular eye muscle surgery, the biomaterial can be contacted with one side or portion of the eye muscle, or can be wrapped or formed around the muscle. In a specific embodiment, the eye surgery is performed to correct strabismus. In specific embodiments, the eye muscle surgery is a resection, recession, advancement, myectomy, myotomy, tenectomy, or tenotomy. In another specific embodiment, the biomaterial is contacted with the eye muscle in an orbital socket at the point of attachment of the muscle to the orbital socket. In a preferred embodiment, the biomaterial is umbilical cord membrane, and, more preferably, umbilical cord membrane and Wharton's jelly.

### 5.6.5. Vitreoretinal Surgeries

Vitreoretinal surgery is surgery to treat or correct a disease or condition of the vitreous humor or retina. Such conditions include, but are not limited to, diabetic retinopathy; retinal detachment; severe trauma to the eye; macular degeneration; or vascular abnormalities (abnormalities of the blood vessels).

Vitreoretinal surgery includes scleral buckling surgery to repair retinal detachment. Scleral buckling surgery is a method of closing a tear in the retina that allows fluid to come between, e.g., the sensory retina and the retinal pigment epithelium, by bringing the two layers of the retina back together, and getting rid of fluid under the retina. A scleral buckle is produced by, e.g., a piece of silicone sponge, rubber, or semi-hard plastic that is placed against the sclera, “buckling” the sclera inward. This buckling effect on the sclera relieves the pull (traction) on the retina, allowing the retinal tear to settle against the wall of the eye. The buckle effect may cover only the area behind the detachment, or it may encircle the eyeball like a ring. The buckling material is typically sewn to the eye to keep it in place, and is usually left in place permanently.

Another type of vitreoretinal surgery is an anterior vitrectomy, in which the front portion of the vitreous tissue is removed to prevent or treat vitreous humor loss during cataract or corneal surgery, or to remove misplaced vitreous in conditions such as aphakia pupillary block glaucoma. During a vitrectomy operation, tiny incisions are made in the sclera. Using a microscope to look inside the eye and microsurgical instruments, an opthalmological surgeon can, e.g., remove the vitreous and repair the retina through the tiny incisions. Repairs include removing scar tissue or a foreign object if present.

Placental biomaterial or umbilical cord biomaterial can be used in vitreoretinal surgeries to, e.g., substantially or completely cover a discontinuity, e.g., hole or fistula in a tissue of the eye, e.g., the sclera, made during the surgery; to cover stitches or staples used to hold, e.g., buckling implants in place; and the like, so as to effect healing of the tissue. Thus, in another aspect, the invention provide a method of performing a vitreoretinal surgical procedure on an eye, comprising contacting a tissue involved in said surgery with placental biomaterial or umbilical cord biomaterial. In a specific embodiment, the umbilical cord biomaterial is dried umbilical cord membrane and Wharton's jelly. In a specific embodiment, the surgery comprises making a hole or fistula in a part of the eye, and said contacting comprises substantially or completely covering said hole or fistula. In another specific embodiment, said contacting acts to repair a scleral buckle, e.g., an exposed scleral buckle. In another specific embodiment, said vitreoretinal surgery is a retinal detachment repair surgery. In a more specific embodiment, said retinal detachment repair surgery is laser photocoagulation, pneumatic retinopexy, macular hole repair, partial lamellar sclerouvectomy, partial sclerocyclochorioidectomy, partial lamellar sclerochoroidectomy In another specific embodiment, said vitreo-retinal surgery is anterior vitrectomy, pars plana vitrectomy, or ignipuncture.

### 5.6.6. Oculoplastic Surgeries

Oculoplastic surgeries involve reconstruction of the eye and/or tissues surrounding the eye. A common oculoplastic surgery is a blepharoplasty, which generally designates any surgery to correct a defect in an eyelid, such as the correction of drooping eyelids or removal of fat or excess tissue, e.g., an eyelid reconstruction. Examples of oculoplastic surgeries include, but are not limited to, correction of ptosis, browplasty, eyelid reconstruction, blepharoplasty, ectropion repair, entropion repair, canthal resection, canthectomy, cantholysis, canthopexy, canthoplasty, canthorrhapy, canthotomy, canaliculodacryocystostomy, canaliculotomy, dacryoadenectomy, dacryocystectomy, dacryocystorhiostomy, dacryocystostomy, enucleation of the eye, evisceration or exenteration.

Placental biomaterial is useful in any type of oculoplastic surgery as a patch to be placed (e.g., sutured or glued to) any incision made during such a surgery. In a specific example, the surgery is enucleation, and the umbilical cord biomaterial or placental biomaterial is used to line part or all of the eye socket after removal of the eyeball. In another embodiment, after enucleation, the biomaterial is used to line an orbital implant to replace the removed eye.

Thus, in another aspect, the invention provide a method of performing an oculoplastic surgical procedure comprising contacting an incision made in the surgical procedure with a placental biomaterial or umbilical cord biomaterial, preferably dried umbilical cord membrane, e.g., umbilical cord membrane and Wharton's jelly. In a specific embodiment, said oculoplastic surgery is a blepharoplasty, e.g., eyelid reconstruction. In a specific embodiment, the biomaterial used in an oculoplastic surgery is about 400 nm in thickness. In another specific embodiment, said oculoplastic surgery is reconstruction of a exposed orbital implant.

### 5.6.7. Other/Miscellaneous Surgeries and Procedures

Other ocular surgeries include, but are not limited to, ciliarotomy, ciliectomy, ciliotomy, corectomedialysis, corectomy, corelysis, coremorphosis, coreplasty, coreoplasty, pupillomydriasis, cyclectomy, cyclectomy, iridectomesodialysis, iridodialysis, iridesis, iridocorneosclerectomy. The placental biomaterial or umbilical cord biomaterial, e.g., fresh or dried umbilical cord membrane or umbilical cord membrane can be used as a patch or tissue support in any of these surgeries in the same manner as for the surgeries described above.

In a specific embodiment, surgery or other procedures are performed to correct, repair or ameliorate trauma to the eye. In various embodiments, the trauma can be a puncture (that is, formation of a hole in a part of the eye), laceration, abrasion, burn or thermal damage, and the like. Generally, the surgery or other procedure comprises contacting the site of trauma with one or more pieces of biomaterial. The biomaterial can be held in place by, e.g., sutures or a tissue adhesive suitable for use in the eye.

### 5.7. Kits

The present invention further provides kits comprising dried placental biomaterial or umbilical cord biomaterial for use in ocular surgery in an appropriately labeled container. Such kits comprise one or more pieces of such biomaterial suitable for ocular use, for example, one or more pieces of umbilical cord biomaterial that are at least 0.25 cm×0.25 cm to about 1 cm×1 cm in size. Preferably, each piece is individually wrapped in, e.g., a peel pouch or other easily manipulable and openable packaging. The kit can additionally comprise one or more solutions for rehydrating, e.g., dried umbilical cord biomaterial prior to use. In certain embodiment, the invention provides a kit for performing a scleral buckle surgery, comprising a piece of placental biomaterial or umbilical cord biomaterial, and a composition for creating a buckle in the sclera of an eye, in a container. In one embodiment, the kit comprises a set of instructions for an end user, e.g. medical personnel or opthalmological surgeon, which instructions can comprise directions on handling and rehydrating the biomaterial, and, in certain embodiments, detailed instructions on the use of the biomaterial for one or more specific ocular surgeries.

The kit can comprise one or more compositions that can be used to fasten or fix the biomaterial in place in or on the eye, eyelid, or surrounding tissue. Such a composition can be, for example, a tissue glue or tissue adhesive, e.g., a cyanoacrylate (e.g., N-butyl-2-cyanoacrylate, HISTOACRYL™), fibrin glue, fibrinogen glue, a hydrogel tissue glue, chondroitin sulfate aldehyde, natural proteins (e.g., BD Cell-Tak™ (BD Biosciences)), and the like. Thus, in one embodiment, the kit comprises one or more pieces of placental biomaterial or umbilical cord biomaterial and a container of a tissue glue or tissue adhesive.

In another embodiment, the composition in the kit used to fasten or fix the biomaterial in place is a suture. Such sutures can be, for example, resorbable or non-resorbable sutures.

The container in which kit components are handled and sold is preferably labeled per applicable Food and Drug Administration standards.

### 6. EXAMPLES

### 6.1. Example 1

### Production of Umbilical Cord Biomaterial

The following example demonstrates one method of preparing umbilical cord biomaterial.

Materials and Equipment. The following items were obtained and, where appropriate, sterilized: human placenta (less than 48 hours old at the start of processing); surgical clamps/hemostats; scissors; scalpels; tweezers; Halsted mosquito; Adson bayonet forceps; grooved directors; cell scraper; autoclaved gauze; stainless steel rinsing trays; stainless steel cups; stainless steel processing trays. 0.9% NaCl solution; sterile water; specimen containers; personal protective equipment (including sterile and non-sterile gloves); certified clean room; decellularizing solution (0.5% deoxycholic acid solution); rocking platform (VWR Model 100); timer (VWR TRACEABLE® model); disinfected silicone grid; PVC wrap film; vacuum pump (Schuco-Vac 5711-130); heat dryer (BioRad Model 583); sterile cutting board; pouches for packaging (COT-360, 361, 362); stainless steel ruler; TRACEABLE® Digital Thermometer (Model 61161-364, Control Company); Accu-Seal Automatic Sealer (Accu-Seal, Model 630-1 B6 or 730-16B) with air compressor; and waterproof resealable bags (CCT-03S).

Procedure. A sterile field was set. The placenta was removed from the transport container and placed into a sterile stainless steel tray. Using surgical damps and scissors, the umbilical cord was cut off approximately 2 inches from the placental disc. The umbilical cord was rinsed with sterile 0.9% NaCl solution as many times as necessary to remove as much blood as possible; optionally, fingers were used to squeeze remaining blood from vessels. The umbilical cord was optionally placed in a separate sterile container cup prefilled with sterile 0.9% NaCl solution, if the cord did not have to be processed immediately. The harvested umbilical cord was placed in a refrigerator at 4° C. until use. The placental disk was placed back into the transport container to be utilized for other projects, or discarded.

The umbilical cord was processed as follows. The umbilical cord was removed from the specimen container, and squeezed to remove any remaining blood from vessels prior to introducing the umbilical cord to a processing tray. The umbilical cord was placed into a sterile stainless steel processing tray, and cut into segments 12 to 15 cm in length. The umbilical cord vein was then located for each segment, and canalized using an Adson bayonet forceps or grooved director. The vein and umbilical cord were then cut longitudinally, using scissors, until both the vein and umbilical cord were fully open. The umbilical cord, was placed on the processing tray with the opened vein side facing upward. The umbilical cord and vein were then bluntly dissected longitudinally between the vein wall and the umbilical cord wall with sterile tweezers or mosquito clamps. When both sides were separated, the vein was carefully removed. After vein removal, the two arteries were located and removed in the same manner. Depending on the purpose of the study, the resulting umbilical cord membrane (biomaterial) was placed in either in saline solution or in 1% deoxycholic acid solution (a decellularizing solution) and stored at 4° C. until serological testing results become available.

Storage and Quarantine of Umbilical Cord Membrane. The umbilical cord membrane, obtained as outlined above, was kept in sterile 0.9% saline solution or 1% deoxycholic acid solution for 10-20 days at 4° C. until serological test results, if ordered, were available. Saline solution, where used, was changed every 3 days. 1% deoxycholic acid solution, when used, was changed every 5 days.

Umbilical Cord Membrane Cleaning and Rinsing. A sterile field was set with a new set of sterilized trays as above. The Umbilical cord membrane was removed from the refrigerator and placed into a stainless steel processing tray. Sterile 0.9% saline solution is added to cover the bottom of the tray. All, or substantially all, residual deoxycholic solution, where used, was removed, and remaining cells and debris were removed from both sides of the tissue using a cell scraper and sterile tweezers. Sterile 0.9% saline solution was used as needed to aid in removal of the cells and debris. The umbilical cord membrane was rinsed three times in a separate stainless steel rinsing tray filled with sterile 0.9% saline solution. The saline solution was changed between each cleaning step. The umbilical cord membrane was then placed into a new sterile specimen container containing about 150 mL saline solution, and placed on a rocking platform for agitation for 5 minutes at setting #6. The scraping and rinsing steps were repeated once as necessary. The umbilical cord membrane was then placed into a sterile specimen container containing 150 mL sterile water, and placed on a rocking platform for agitation for 20 minutes at setting #6. This rinsing step was repeated three times.

Drying The Umbilical Cord Membrane. A TYVEK® sheet was placed onto a stainless steel processing tray. The cleaned umbilical cord membrane segments were removed from the specimen container one piece at a time, and excess fluid was gently squeezed out. The membrane segments were then placed on the surface of the TYVEK® sheet, epithelium side up, and gently stretched until flat. The membrane was then dried at about 50° C.±1.0° C. in a vacuum dryer. Sterile gauze was placed on the drying platform of the vacuum dryer, covering an area slightly larger than the area of the TYVEK® sheet. The total thickness of the gauze layer did not exceed the thickness of one folded 4×4 gauze. A sheet of silicone framing mesh was placed on top the gauze, smooth side up. The TYVEK® sheet with the tissue was then placed on the heat dryer platform on top of the silicone mesh. Another TYVEK® sheet was then placed on top of the tissue. A piece of PVC wrap film was then cut large enough to cover the entire drying platform, and pulled so that the film pulled tightly against the TYVEK® sheet (that is, was “sucked in” by vacuum) and so that there were no air leaks and no wrinkles over the tissue area). The vacuum pump was then set to approximately −22 inches Hg, and heat/vacuum drying was allowed to proceed for a total of about 120 minutes. Approximately 30-45 minutes into the drying process, the sterile gauze layer was replaced.

A new sterile field was set with a sterilized drying kit and cutting board. With the pump still running, the plastic film was removed from the TYVEK®, and the sheet and tissue were placed on a cutting board with the epithelium side of the tissue facing upward. The dried membrane (now umbilical cord biomaterial) was then gently removed from the TYVEK® sheet. The biomaterial segments were then cut with a scalpel into segments of a specified size, typically 2×2 cm or 1×1 cm. The dried, sized umbilical cord biomaterial was then placed and sealed into a peel-pouch package.

### 6.2. Example 2

### Production of Umbilical Cord Biomaterial Laminate

Objective: To increase the size of a sheet of umbilical cord biomaterial for hernia repair.

Materials and Methods: All cited dimensions are approximate. Umbilical cord membrane from a 23.5 hour-old placenta was collected and processed as in Example 1 up to the point of drying. The final size of the membrane was approximately 35 cm by 4 cm. The membrane was cut into three pieces approximately 10 cm long. The pieces were arranges so as to overlap by about 2 cm on the long edge, and were dried at 50° C. between two sheets of TYVEK®.

Results: The biomaterial comprising laminated membrane thus obtained was approximately 10 cm by 10 cm. The sections did not separate upon rehydration in saline for 72 hours.

Umbilical cord membrane can also be laminated by placing two or more pieces of the biomaterial, interior (of the umbilical cord) side down, on a substrate in a mounting frame. The laminated membrane is then placed in a gel dryer and dried to substantial dryness (≦about 20% water content by weight) to produce a laminated umbilical cord biomaterial.

Another method of constructing a thicker biomaterial is to laminate intact umbilical cord (including Wharton's jelly but lacking arteries and vein). The intact cord is then processed, e.g., by rinsing, soaking in a solution such as a buffered saline solution, e.g., phosphate buffered saline, or a mild ionic or nonionic detergent solution. The cord is dried in a vacuum dryer to create an intact, double-layer biomaterial. Two or more layers of this double-layer material can be laminated by layering the biomaterials and drying further in a heated vacuum dryer. The drying/dehydration process can be heat drying or any other processes as described below.

### 6.3. Example 3

### Characterization of Dried Umbilical Cord Biomaterial

A study was undertaken to examine biomaterial made of heat dried human umbilical cord membrane (HUC) after sterilization by different doses of gamma irradiation. Samples of HUC were sterilized with 0, 20, 25, 30, or 40 kGy and then examined for water uptake (mass and thickness change), denaturation temperature, and tensile mechanical properties. HUC samples had been incubated for either 10 or 20 days in 1% D-cell (deoxycholic acid) solution during preparation.

### 6.3.1. Water Uptake

Individual samples of HUC for each condition (n=3) were weighed on a microbalance. Samples were then incubated in 10 mL of phosphate buffered saline at 37° C. for 1 hour. Samples were removed from the PBS and blotted dry a minimum of three times with a KIMWIPE® tissue. The samples were again weighed on a microbalance. The percentage water uptake ([wet weight (Ww)−dry weight (Wd)]/Wd*100) and equilibrium water content ([Ww−Wd]/Ww*100) were calculated.

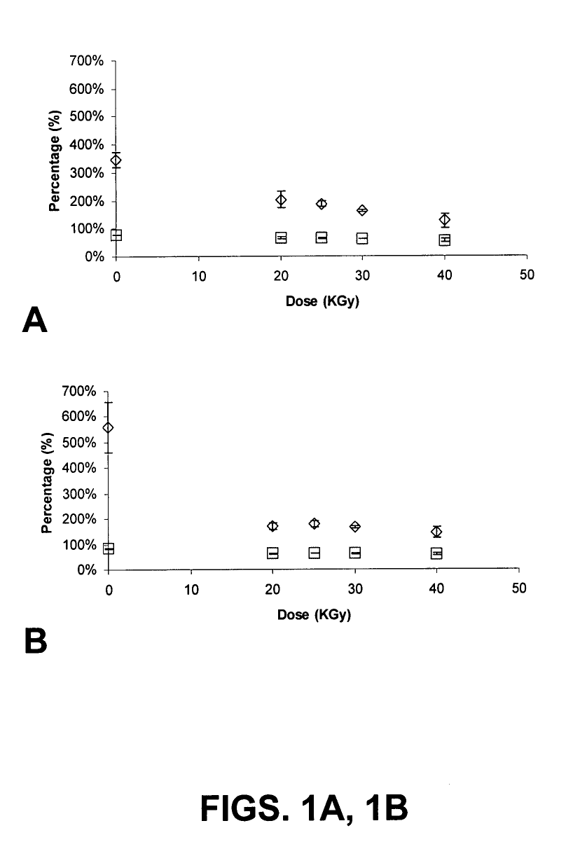

FIG. 1 summarizes the results of the rehydration for HUC incubated for 10 and 20 days. The initial water uptake of the control (nonsterilized samples) was much higher for HUC incubated for 20 days than for HUC incubated for 10 days, possibly due to the loosening of the membrane proteins by the detergent effect of the deoxycholic acid in the D-cell solution. Water uptake and equilibrium water content closely matched for the 10 and 20 days samples that were sterilized at all radiation doses. There was a linear decrease in the water uptake and the equilibrium water content of both sets of samples with increasing radiation dose. Even at the highest radiation dose, the membranes took up at least their own weight in water.

### 6.3.2. Changes in Thickness

Individual samples of HUC for each condition (n=8) were mounted in squares of vellum paper so that the membrane could be easily handled during and after hydration. The thickness was measured in three locations for each sample and averaged. Samples were then incubated in 10 mL of phosphate buffered saline at 37° C. for 1 hour. Thickness measurements were repeated after hydration.

Overall, the average dry thickness of the HUC was ˜70 μm, with the 10 and 20 day samples having average thickness of 57 and 86 μm respectively (Table 1 and Table 2). There appeared to be no correlation between radiation dose and dry thickness of the HUC. After rehydration, there was a marked difference in thickness between the sterilized and non-sterilized samples. There was little difference in the rehydrated thickness between the 10 and 20 day samples. Without wishing to be bound by any theory or mechanism, the difference observed can be due to cross-linking of collagen molecules caused by irradiation. There appeared to be a decrease in the magnitude of the thickness change upon rehydration with increasing dose; this effect was more pronounced with the 10 day samples.

TABLE 1
Changes in thickness of 10 day incubated HUC during hydration

| Dose | | | | | increase | |
|---|---|---|---|---|---|---|
| (KGy) | dry (um) | SD | wet (um) | SD | (%) | SD |
| 0 | 61 | 23 | 243 | 82 | 316% | 109% |
| 20 | 44 | 10 | 107 | 20 | 147% | 19% |
| 25 | 58 | 24 | 132 | 40 | 134% | 28% |
| 30 | 67 | 22 | 125 | 45 | 87% | 32% |
| 40 | 57 | 23 | 106 | 46 | 91% | 36% |

TABLE 2
Changes in thickness of 20 day incubated HUC during hydration

| Dose | | | | | increase | |
|---|---|---|---|---|---|---|
| (KGy) | dry (um) | SD | wet (um) | SD | (%) | SD |
| 0 | 88 | 22 | 279 | 51 | 240% | 123% |
| 20 | 76 | 22 | 149 | 26 | 106% | 48% |
| 25 | 85 | 50 | 130 | 21 | 83% | 61% |
| 30 | 61 | 23 | 138 | 51 | 125% | 31% |
| 40 | 121 | 32 | 209 | 46 | 77% | 36% |

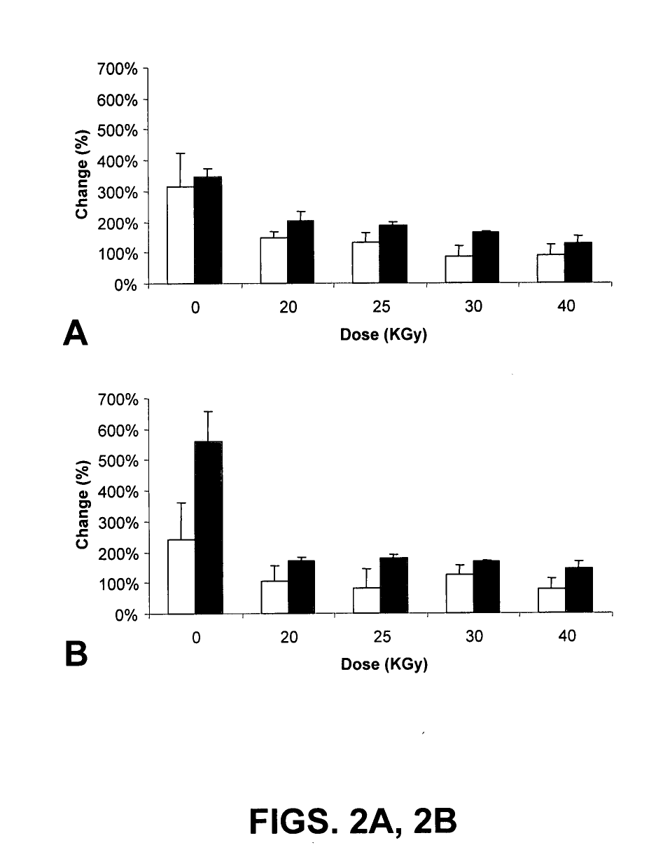

When the change in thickness of the membranes was compared to the water uptake (FIG. 2), there was a loose correlation between the amount of water taken up and the increase in thickness. The magnitude of the water uptake and the change in thickness both decreased with increasing gamma radiation dose. There was a stronger correlation between water uptake and thickness change for samples incubated for 10 days than those incubated for 20 days. This is due partially to the fact that the samples incubated for 20 days showed less difference between samples, but greater variability within a set of samples.

### 6.3.3. Denaturation Temperature

Individual samples of HUC for each condition (n=3) were incubated in 10 mL of phosphate buffered saline at 37° C. for 1 hour. Samples were removed from the PBS and blotted dry a minimum of three times with a KIMWIPE® tissue. The samples were sealed in aluminum hermetic differential scanning calorimeter (DSC) pans and tested in a TA Instruments modulated DSC (Q1000) in standard mode from 5-110° C. at 10° C./min. TA Instruments' “Universal Analysis” software was used to calculate the onset and peak values of the denaturation point of the membranes.

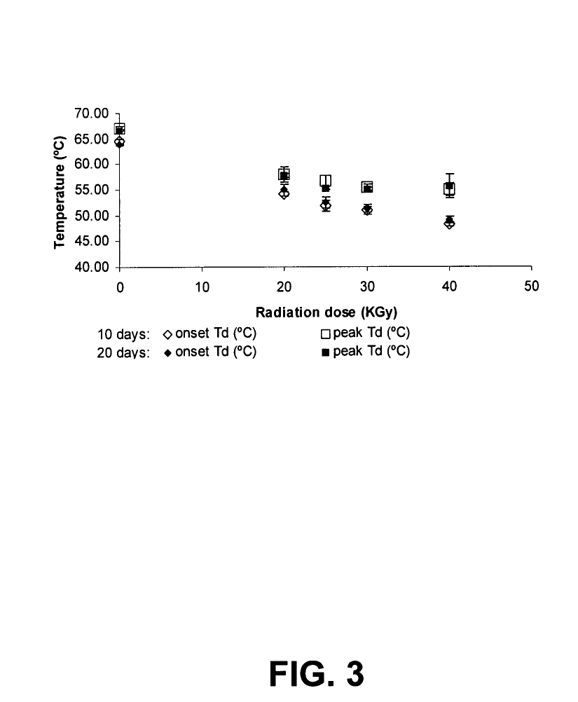

FIG. 3 graphically summarizes the denaturation temperature results for the HUC samples. The results are identical for samples incubated for 10 and 20 days. Onset and peak temperatures were only a few degrees different for each of the samples, and both onset and peak temperatures decreased linearly with increasing radiation dose. There was very low variability in the results.

Time of incubation in D-cell solution did not affect the denaturation temperature. There was a linear decrease in the onset and peak denaturation temperatures with increasing radiation dose for both the 10 and 20 day samples. From the denaturation data, there appeared to be no difference between soaking HUC for 10 or 20 days in 1% D-cell solution.

### 6.3.4. Suture Pull-Out Strength

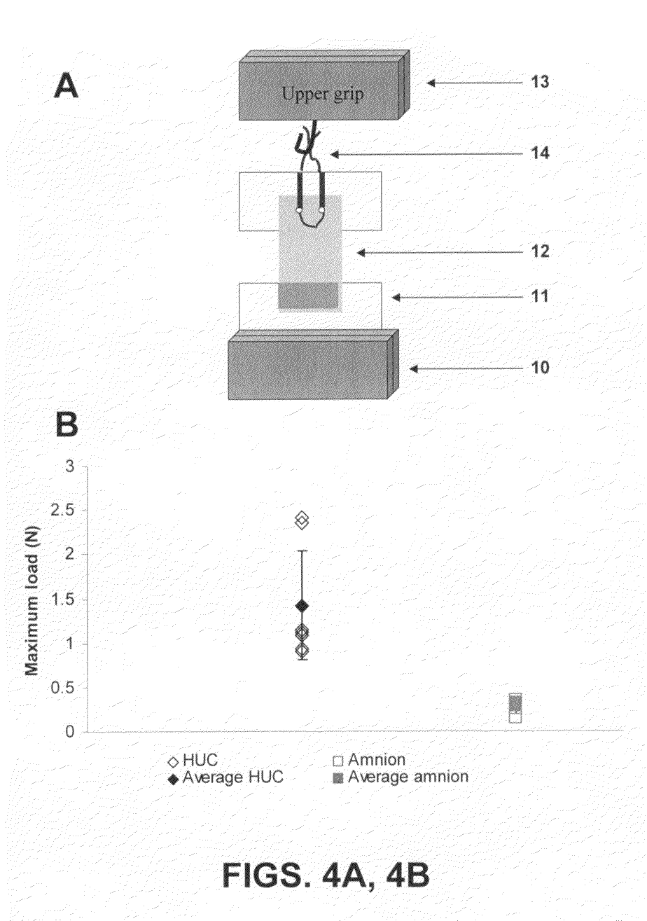

The umbilical cord biomaterial was determined to have a superior suture pull-out strength compared to dried human amniotic membrane. In a test similar to that described in Section 6.3.4, above, one short side of a 1×2 section of umbilical cord biomaterial was glued to vellum paper, and the other short side sutured to a second piece of vellum paper, as depicted in FIG. 4A. The pieces of vellum paper were held by grips, and a load was applied to the suture at a rate of about 12.7 mm/min. The umbilical cord biomaterial demonstrated an average pull-out resistance of about 1.4 Newtons (N), with a range of about 0.75 N to about 2.4 N, while the dried amniotic membrane demonstrated a pull-out resistance averaging about 0.3 N. See FIG. 4B.

### 6.4. Example 4

### Drainage Implant/Valve Implantation

This Example demonstrates the use of dried umbilical cord biomaterial in the implantation of a drainage implant or valve in glaucoma surgery.

A patient presents with glaucoma resolvable by implantation of a drainage implant or valve. After appropriate anesthetization, a silicone drainage tube is inserted into the anterior chamber of the eye. The tube is connected to an external silicone rubber explant designed to maintain space between the sclera and conjunctiva. The exposed portion of the tube is covered with a patch made from dried umbilical cord membrane, comprising Wharton's jelly, that has been cut to 0.5 cm×0.5 cm and rehydrated in normal saline solution. The patient is monitored for the following six months to determine that no deterioration of the outer portion of the tube has occurred.

### 6.5. Example 5

### Sealing of a Flap Perforation in Glaucoma Surgery

This Example demonstrates the use of dried umbilical cord biomaterial in the repair of a flap perforation resulting from glaucoma surgery.

A patient presents with glaucoma resolvable by a glaucoma filtering operation. A trabeculectomy (posterior lip sclerectomy) is performed in which part of the trabecular network is excised. An incision is made approximately 8 mm behind the limbus, and the conjunctiva and Tenon's capsule are undermined to the Tenon's scleral reflection. In an undesirable development, a perforation of the conjunctiva at the anterior limit of the undermined area occurs at this point. The operation proceeds through sclerectomy and iridactomy, and the perforation is sealed as follows. The original incision is sealed by suturing the anterior lip to the posterior lip of Tenon's capsule using 4-0 double-armed black silk suture. The area between the incision and limbus is painted with iodine solution to ablate the surface epithelium. A piece of dried umbilical cord membrane comprising Wharton's jelly, measuring 1 cm×0.5 cm, is placed over the perforation in the conjunctiva, and is sutured in place. Air is injected using a 30-gauge needle, beneath the original flap, creating a bleb that fills the anterior chamber. The eye is dressed with steroid ointment to avoid scarring of the bleb.

### EQUIVALENTS

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

What is claimed is:

1. A method of performing an ocular surgical procedure that involves a structure of an eye or tissue adjacent to an eye, comprising contacting said structure of the eye or tissue adjacent to the eye with umbilical cord membrane or umbilical cord biomaterial.

2. The method of claim 1, wherein said contacting comprises using said umbilical cord membrane as a patch over a discontinuity in said structure.

3. The method of claim 2, wherein said discontinuity is an incision made during said ocular surgical procedure.

4. The method of claim 3, wherein said ocular surgical procedure is creation of a scleral buckle.

5. A kit comprising:
a. an isolated umbilical cord membrane or umbilical cord biomaterial; and
b. instructions for use of said isolated umbilical cord membrane or umbilical cord biomaterial in performing an ocular surgical procedure, wherein said ocular surgical procedure comprises contacting said structure of the eye or tissue adjacent to the eye with said umbilical cord membrane or umbilical cord biomaterial.

6. A method of performing an ocular surgical procedure that involves a structure of an eye or tissue adjacent to an eye, comprising contacting said structure of the eye or tissue adjacent to the eye with umbilical cord membrane or umbilical cord biomaterial, wherein said ocular surgical procedure is glaucoma surgery.

7. The method of claim 6, wherein said glaucoma surgery comprises implantation of a drainage implant or tube shunt.

8. The method of claim 7, wherein said umbilical cord membrane additionally contacts said drainage implant or said tube shunt.

9. The method of claim 8, wherein said umbilical cord membrane or umbilical cord membrane substantially prevents deterioration of said silicone tube for at least six months.

10. The method of claim 6, wherein said glaucoma surgery is trebeculoplasty, iridotomy, iridactomy, a filtering surgery, anterior sclerotomy, sclerostomy, penetrating trabeculotomy, non-penetrating trabeculotomy, trepanotrabeculectomy, goniotomy, cyclotomy, cycloanemization, cyclocryotherapy, cyclocryopexy, iridenclesis, iridocyclectomy, iridosclerotomy, corenclisis or goniocurretage.

11. A method of performing an ocular surgical procedure that involves a structure of an eye or tissue adjacent to an eye, comprising contacting said structure of the eye or tissue adjacent to the eye with umbilical cord membrane or umbilical cord biomaterial, wherein said ocular surgical procedure is cataract surgery.

12. The method of claim 11, wherein said cataract surgery comprises phacoemulsification of a cataract in said eye.

13. The method of claim 12 wherein said contacting comprises repair of a phacoemulsification burn.

14. The method of claim 1, wherein said ocular surgical procedure is a trabulectomy.

15. The method of claim 1, wherein said ocular surgical procedure is implantation of an orbital implant.

16. The method of claim 1, wherein said ocular surgical procedure is corneal surgery.

17. The method of claim 16, wherein said corneal surgery changes the shape of the cornea.

18. The method of claim 17, wherein said corneal surgery is photorefractive keratectomy (PRK), laser-assisted sub-epithelial keratectomy (LASEK), laser-assisted in situ keratomileusis (LASIK), automated lamellar keratoplasty (ALK), laser thermal keratoplasty (LTK), or conductive keratoplasty (CK).

19. A method of performing an ocular surgical procedure that involves a structure of an eye or tissue adjacent to an eye, comprising contacting said structure of the eye or tissue adjacent to the eye with umbilical cord membrane or umbilical cord biomaterial, wherein said ocular surgical procedure is vitreo-retinal surgery.

20. The method of claim 19, wherein said vitreo-retinal surgery is an anterior vitrectomy, pars plana vitrectomy, ignipuncture, macular hole repair, partial lamellar sclerouvectomy, or posterior sclerotomy.

21. The method of claim 1, wherein said ocular surgical procedure is retinal surgery.

22. The method of claim 1, wherein said ocular surgical procedure is an eye muscle surgery.

23. A method of performing an ocular surgical procedure that involves a structure of an eye or tissue adjacent to an eye, comprising contacting said structure of the eye or tissue adjacent to the eye with umbilical cord membrane or umbilical cord biomaterial, wherein said ocular surgical procedure is oculoplastic surgery.

24. The method of claim 23, wherein said oculoplastic surgery is browplasty, eyelid reconstruction, blepharoplasty, ectropion repair, entropion repair, canthal resection, canthectomy, cantholysis, canthopexy, canthoplasty, canthorrhapy, canthotomy, canaliculodacryocystostomy, canaliculotomy, dacryoadenectomy, dacryocystectomy, dacryocystorhiostomy, dacryocystostomy, enucleation of the eye, evisceration or exenteration, or repair of an exposed orbital implant.

25. The method of claim 1, wherein said ocular surgical procedure is ciliarotomy, ciliectomy, ciliotomy, corectomedialysis, corectomy, corelysis, coremorphosis, coreplasty, coreoplasty, pupillomydriasis, cyclectomy, cyclectomy, iridectomesodialysis, iridodialysis, iridesis or iridocorneosclerectomy.
